(19) United States
(12) Patent Application Publication
(10) Pub. No.: US 2026/0176625 A1
(43) Pub. Date: Jun. 25, 2026

(54) LIPID NANOPARTICLES WITH BLEBS HAVING IMPROVED TRANSFECTION EFFICIENCY

(71) Applicants: The University of British Columbia, Vancouver (CA); NanoVation Therapeutics Inc., Vancouver (CA)

(72) Inventors: Pieter R. CULLIS, Vancouver (CA); Jerry LEUNG, Vancouver (CA); Miffy Yok Han CHENG, Vancouver (CA); Jayesh A KULKARNI, Vancouver (CA); Dominik WITZIGMANN, Vancouver (CA); Genc BASHA, Vancouver (CA)

(21) Appl. No.: 19/125,459

(22) PCT Filed: Oct. 31, 2023
(86) PCT No.: PCT/CA2023/051451

§ 371 Date: Apr. 29, 2025

Related U.S. Application Data
(60) Provisional application No. 63/420,814, filed on Oct. 31, 2022.
(60) Provisional application No. 63/444,113, filed on Feb. 8, 2023.

(51) Int. Cl. C12N 15/113; A61K 9/51; A61K 48/00

(52) U.S. Cl. CPC C12N 15/113; A61K 9/5123; A61K 48/00

(57) ABSTRACT

Provided herein is a method for preparing the nanoparticle comprising: (i) combining an aqueous phase comprising the nucleic acid with an organic solvent-lipid mixture comprising lipids, wherein the lipids comprise an ionizable lipid and at least one helper lipid and optionally a hydrophilic-polymer lipid conjugate; wherein the aqueous phase comprises a buffer having a concentration of at least 100 mM and has an aqueous phase pH that is lower than a pKa of the ionizable lipid such that the ionizable lipid is substantially charged; wherein the lipid nanoparticle is formed during or subsequent to the combining; and (ii) exchanging a solution external to the lipid nanoparticle with a higher pH solution, thereby producing the nucleic-acid lipid nanoparticle, wherein the nucleic-acid lipid nanoparticle comprises one or more bleb compartments.

### CROSS-REFERENCE TO RELATED APPLICATIONS

This application claims the benefit of priority from U.S. provisional patent application No. 63/420,814 filed on Oct. 31, 2022 and U.S. provisional patent application No. 63/444,113 filed on Feb. 8, 2023, each of which are incorporated herein by reference in their entirety.

### TECHNICAL FIELD

The present disclosure is directed to a nucleic acid-lipid nanoparticle, methods of making such nanoparticle and a method for screening for nucleic acid-lipid nanoparticles having improved transfection potency.

### BACKGROUND

Lipid nanoparticle (LNP) systems are playing a pivotal role in enabling therapeutics based on short interfering RNA (siRNA) for silencing pathological genes and mRNA for expressing therapeutic proteins (Cheng et al., 2022, Mol Pharm, 19:1663-1668). Two major examples are Onpattro™ (Akinc et al., 2019, Nat Nanotechnol, 14:1084-1087), an LNP formulation of siRNA to treat transthyretin induced amyloidosis that was approved by the FDA in 2018 and the COVID-19 mRNA vaccines that have received regulatory approval in many jurisdictions worldwide (Polack et al., 2020, N Eng J Med, 383:2603-2615). There are many more LNP RNA therapeutics in development with potential applications ranging from treating atherosclerosis (Musunuru et al., Nature, 593:429-434) to heart failure (Rurik et al., 2022, Science, 375:91-96).

LNP formulations of siRNA and mRNA for liver targets or vaccine applications are composed of ionizable cationic lipids, phospholipids, cholesterol, and polyethylene glycol (PEG)-lipids, usually in the approximate molar ratios of 50/10/38.5/1.5. It is widely recognized that the ionizable lipid component plays a role in determining transfection potency and considerable effort has gone into synthesizing progressively optimized ionizable lipids. For example, the gene silencing potency of LNP containing siRNA to silence FVII in hepatocytes in the liver following an i.v. injection ranges from an ED50 of ˜40 mg/kg using DODAP (Semple et al., 2010, Nat Biotechnol, 28:172-176), the first ionizable cationic lipid employed to formulate LNP (Semple et al., 2001, Biochim Biophys Acta, 1510:152-166), to ˜1 mg/kg for DLinDMA (Zimmermann et al., 2006, Nature, 441:111-114) to 0.01 mg/kg for DLinKC2DMA (KC2) (Semple et al., 2010, Nat Biotechnol, 28:172-176) and finally to 0.005 mg/kg for DLinMC3DMA (MC3) (Jayaraman et al., 2012, Angew Chem Int Ed Engl, 51:8529-8533). MC3 is the ionizable lipid employed in Onpattro™. In the case of mRNA, these synthetic efforts have continued using gene expression in the liver as the measure of transfection potency, resulting in lipids such as ALC-0315 and SM-102. LNP systems incorporating ALC0315 and SM102, which are the ionizable lipids employed in the Pfizer/BioNTech and Moderna COVID-19 mRNA vaccines respectively, exhibit ten-fold or higher gene expression in the liver than can be achieved with LNP containing MC3.

While significant advances have been made in enhancing the transfection potency of LNPs by optimizing the ionizable lipid, there is an ongoing need for further improvements. Such improvements will further expand the utility of LNPs to treat or prevent a wider range of disorders or improve the efficacy of existing LNP formulations.

### SUMMARY

The present disclosure addresses the ongoing need to improve the potency of a lipid nanoparticle (LNP) for the delivery of nucleic acid and/or provides useful alternatives to known nucleic acid-LNP formulations.

In particular, the present disclosure is based on the discovery that nucleic acid-lipid nanoparticle formulations comprising one or more “bleb” structures as described herein provide surprising improvements in transfection potency both in vitro and in vivo relative to a baseline LNP lacking such structures.

In some embodiments, the bleb structure is characterized as a protrusion in the lipid nanoparticle that contains an aqueous compartment comprising the nucleic acid as observed by cryo-TEM.

As described further herein, such bleb structures may be induced by increasing the concentration of the buffer containing the nucleic acid during formulation of the LNP and/or by selection of a suitable ionizable lipid that results in such bleb formation. Without being limited by theory, the improvements in potency may be the result of nucleic acid contained in the aqueous compartment of the bleb being lipid-dissociated due to the high ionic strength of the buffer present in the compartment.

According to one aspect of the disclosure, there is provided a nucleic acid-lipid nanoparticle comprising: a lipid layer surrounding at least two compartments, the first compartment being an aqueous bleb compartment comprising a buffer and a nucleic acid, the bleb compartment having a buffer ionization strength causing the nucleic acid to be lipid-dissociated; and a second hydrophobic compartment comprising an ionizable lipid in neutral form.

In a further aspect of the disclosure, there is provided a nucleic acid-lipid nanoparticle comprising a lipid layer and at least one bleb structure protruding from the lipid layer, the bleb structure having an internal buffer concentration that is at least 50 mM, least 100 mM, at least 150 mM, at least 200 mM, at least 250 mM, at least 275 mM or at least 300 mM and having a pH above the pKa of the ionizable lipid.

According to a further aspect of the disclosure, there is provided a method for preparing a nucleic-acid lipid nanoparticle, the method comprising: (i) combining an aqueous phase comprising the nucleic acid with an organic solvent-lipid mixture comprising lipids, wherein the lipids comprise an ionizable lipid and at least one helper lipid; wherein the aqueous phase comprises a buffer having a concentration of at least 100 mM and has an aqueous phase pH that is lower than a pKa of the ionizable lipid such that the ionizable lipid is substantially charged; wherein the lipid nanoparticle is formed during or subsequent to the combining; and (ii) exchanging a solution external to the lipid nanoparticle with a higher pH solution, thereby producing the nucleic-acid lipid nanoparticle that comprises one or more bleb compartments.

Current methods of screening for LNP in vivo activity rely on in vivo studies, such as expression of luciferase in liver following i.v. administration. Such studies are time-consuming and costly. Accordingly, the inventors have determined that screening for the ability to induce bleb structures in nucleic acid-LNPs using cryo-TEM is a very useful adjunct to in vivo screening. Similarly, such screening could streamline the design of in vitro studies.

Thus, according to a further aspect of the disclosure, there is provided a method for screening a lipid nanoparticle preparation for in vitro or in vivo potency, the method comprising: (i) producing a lipid nanoparticle preparation incorporating a nucleic acid; (ii) determining whether at least a portion of lipid nanoparticles in the preparation comprise bleb structures, wherein the bleb structures enclose an aqueous compartment comprising the nucleic acid; and (iii) if the bleb structures are present in the preparation, identifying the particles for further assessment in an in vitro and/or in vivo potency assay.

According to a further aspect of the disclosure, there is provided a lipid nanoparticle preparation prepared by any one of the foregoing methods.

### BRIEF DESCRIPTION OF THE FIGURES

FIG. 1 shows that formulation of LNP luc mRNA systems using high (300 mM) concentration buffers (pH 4) leads to increased bleb structures for LNP mRNA formulations containing KC2 and improved transfection potencies. FIG. 1a shows LNP of composition: KC2/DSPC/cholesterol/PEG-DMG at 50/10/38.5/1.5 (mol/mol) formulated in various pH 4 buffers: 25 mM NaOAc, 300 mM NaOAc, 300 mM Na-citrate Phosphate (CitPhos), 300 mM Na-citrate (left to right); FIG. 1b shows luminescence of Huh7 cells incubated for 24 hr with LNP luc mRNA systems formulated with the foregoing pH 4 buffers (mean±SEM, n=3). Data was analyzed through a two-way ANOVA, ^{ns} non-significance, ** P<0.01 and **** P<0.0001. FIG. 1c shows the percentage of bleb and non-bleb structural morphology of LNP luc mRNA formulated with the foregoing pH 4 buffers (mean±SEM, n=3). FIGS. 1D-G shows cryo-TEM images of LNP luc mRNA formulated with various pH 4 buffers, namely d, 25 mM NaOAc, e, 300 mM NaOAc, f, 300 mM CitPhos, and g, 300 mM Na-citrate. The white arrows point to LNP exhibiting normal solid core structure whereas the grey arrows indicate bleb structures. White scale bar for enlarged images=100 nm.

FIG. 2 shows that formulation of LNP luc mRNA using high (300 mM) concentration Na-citrate buffer (pH 4) leads to increased bleb structures for LNP mRNA formulations containing a variety of ionizable cationic lipids. LNP of composition. FIGS. 2A-C show ionizable lipid/DSPC/cholesterol/PEG-DMG (50/10/38.5/1.5 mol/mol) formulated in 25 mM NaOAc, and FIG. 2D-F shows the same formulations in 300 mM Na-citrate. The ionizable lipid was KC2, ALC-0315 and SM-102 as indicated (left to right).

FIG. 3 shows that increasing the formation of bleb containing mRNA with higher concentration buffers increase the sizes of LNP and the number of mRNA each bleb contains. LNP of composition: KC2/DSPC/cholesterol/PEG-DMG (50/10/38.5/1.5 mol/mol) formulated in A) 25 mM NaOAc, B) 300 mM NaOAc, C) 300 mM Na-citrate Phosphate, D) 300 mM Na-citrate. E) Number of mRNA copies associated with blebs per LNP based on cross sectional area of LNP formulated in various buffers.

FIG. 4 shows the in vitro transfection potency of LNP luc mRNA systems containing DLinkC2DMA is dependent on the concentration and/or type of buffer (pH 4) employed during formulation. FIG. 4A shows luminescence of HuH7 cells after incubated with LNP luc mRNA systems containing KC2 (see Methods) and that were formulated with the buffers indicated (25 mM NaOAc, 300 mM NaOAc, 300 mM Na-citrate Phosphate (CitPhos), and 300 mM Na-citrate, left to right). In order to show that the increases in potency are not due to increased cellular uptake, uptake into cells was monitored by the mean fluorescence intensity of DiI from LNP formulated with the buffers indicated and the results are shown in FIGS. 4B and 4C.

FIG. 5 shows the fold improvement in transfection potency in vitro arising from 300 mM Na-citrate buffers depends on the ionizable cationic lipid employed. FIG. 5A shows luminescence transfection of HuH7 cells treated with LNPs of different ionizable lipids and ionic strength buffer at 24 hours post incubation, where the 1^{st}, 3^{rd} and 5^{th} groups of 4 bars are 25 nM NaOAc and the 2^{nd}, 4^{th} and 6^{th} groups of 4 bars are 300 nM Citrate. FIG. 5B shows luminescence transfection efficacy (left y-axis, bars) against the particle diameter as the number mean average (right y-axis, circles), where the 1^{st}, 3^{rd} and 5^{th} bars are 25 nM NaOAc and the 2^{nd}, 4^{th} and 6^{th} bars are 300 nM Citrate.

FIG. 6 shows the in vitro transfection potency of LNP luc mRNA systems containing DLinKC2DMA is positively correlated to the concentration of pH 4 Na-citrate buffer (5-300 mM Na-citrate) and the increasing formation of blebs. LNP of composition: KC2/DSPC/cholesterol/PEG-DMG formulated in 5-300 mM Na-citrate pH 4 buffers. FIG. 6A shows luminescence of HuH7 cells that were incubated with LNP luc mRNA systems containing KC2 (see Methods) (mean±SEM, n=3). FIG. 6B shows size (bars, left y-axis) and encapsulation efficiency (dots, right y-axis) of LNP luc mRNA systems under different buffer conditions as indicated (mean±SEM, n=3). FIG. 6C shows cyro-TEM images of LNP luc mRNA formulated at 5, 25, 50, 150 and 300 mM Na-citrate (pH 7.4).

FIG. 7 shows LNP luc mRNA systems prepared using 300 mM Na-citrate pH 4 buffer exhibit dramatically improved gene expression in vivo compared to LNP luc mRNA systems prepared using 25 mM NaOAc. KC2 LNP luc mRNA and SM-102 LNP luc mRNA systems were formulated using either a 300 mM pH 4 buffer (KC2-300 mM or SM102-300 mM) or a 25 mM NaOAc pH 4 buffer (KC2-25 mM or SM102-25 mM) and administered i.v. at a dose of 0.5 mg mRNA/kg to CD1 mice. Bioluminescence was assayed using IVIS imaging at 3 hr post injection (Panel A), 6 hr post injection (Panel C) and 24 hours post injection (Panel E). Panels B, D and F show the quantified abdominal radiance at 3, 6, and 24 hours post-injection. Values are expressed as mean±SEM (n=3).

FIG. 8 shows LNP luc mRNA systems prepared using 300 mM Na-citrate pH 4 buffer induce significant gene expression in a range of tissues 24 hr post injection. KC2 LNP luc mRNA and SM-102 LNP luc mRNA systems were formulated using either a 300 mM pH 4 buffer (KC2-300 mM or SM102-300 mM) or a 25 mM NaOAc pH 4 buffer (KC2-25 mM or SM102-25 mM) and administered i.v. at a dose of 0.5 mg mRNA/kg to CD1 mice. At 24 hr post injection the mice were sacrificed, organs/tissues removed and bioluminescence assayed by IVIS imaging. A) A strong signal remained in the spleen for LNP prepared using the 300 mM buffer and significant bioluminescence was detected for a variety of organs/tissues for B) KC2 LNP luc mRNA. C) Biodistribution of intravenously injected KC2 LNP luc mRNA and SM-102 LNP luc mRNA (0.5 mg/kg, n=5±SEM) measured through DiD-C18 at 24 h post-injection in CD-1 mice. Four-fold higher spleen uptake can be seen with the KC2-300 mM Na-citrate. D) A strong signal was also observed in the spleen for LNP prepared using the 300 mM buffer SM-102 LNP luc mRNA systems.

FIG. 9 shows the gene silencing potency of LNP siRNA systems is improved when prepared in high concentration pH 4 buffers. Luminescence knockdown of 22Rv1-luc cells treated with LNP-siLuc against luciferase formulated in different ionic strength and buffer at 24 h post incubation.

FIG. 10A depicts generation of bleb structures at high buffer concentrations during dialysis against phosphate buffered saline (PBS), pH 7.4. A. Without being bound by theory, it is believed that at pH 4 all lipids are in condensed bilayer structures such as the concentric multilamellar structure depicted. On incubation against PBS three events occur. First, ionizable lipids in the outside monolayer flip to the inside as they become deprotonated, net neutral molecules that are highly membrane permeable. Once on the inside they become positively charged due to the lower pH. Second, as more protons permeate out through the external bilayer, an increasing proportion of the ionizable lipids in the inner monolayers adopt the neutral, oil form stimulating fusion between apposed bilayers and segregation of the mRNA into a polar compartment. Third, as protons continue to permeate out from the nanoparticle interior, all the ionizable lipids are neutralized and adopt the oil form and the mRNA is segregated into the bleb structure.

FIG. 10B shows that the concentration of pH 4 buffers drives the fusion of small vesicles formed in 25 mM NaOAc pH 4 buffer to form larger structures. Cryo-TEM images of LNP luc mRNA composed of KC2/DSPC/cholesterol/PEG-DMG (50/10/38.5/1.5 mol %). LNP luc mRNA (N/P=6) were formulated in 25 mM NaOAc at pH 4 and first dialyzed against 25 mM NaOAc at pH 4 for 24 hr to remove ethanol. The LNP were then further dialyzed against 300 mM NaOAc, 300 mM CitPhos or 300 mM Na-citrate at pH 4 for an additional 24 hr, which resulted in larger structures.

FIG. 11 shows formulation of LNP luc mRNA systems using high (300 mM) concentration pH 4 Na-citrate buffer leads to fusion and formation of larger LNP systems at pH 4 as compared to LNP prepared in 25 mM NaOAc. LNP luc mRNA (N/P=6) with composition KC2/DSPC/cholesterol/PEG-DMG (50/10/38.5/1.5 mol %) were formulated in 25 mM NaOAc or 300 mM Na-citrate and imaged at pH 4 (FIG. 11A-B), and then were dialyzed against PBS and imaged at pH 7.4 (FIG. 11C-D) FIG. 11E is a schematic of the proposed mechanism whereby blebs form in high ionic strength buffer systems. FIG. 11F shows that LNPs made with 25 mM NaOAc display lower mRNA integrity when incubated in 50% FBS over time as compared to all other 300 mM buffer systems, where from top to bottom, 300 mM Na-citrate, 300 mM Na-citrate Phosphate (CitPhos), 300 mM NaOAc, and 25 mM NaOAc. FIG. 11G shows that 300 mM Na-citrate LNP systems exhibit higher luc mRNA translation in cell-free wheat germ lysate after treating with mRNA incubated in 50% FBS over 24 h (mean±SEM, n=3) at time points 0, 3, 6, and 24 hours (left to right).

FIG. 12 shows that no significant toxicity difference was observed between bleb or non-bleb LNP luc mRNA systems. FIG. 12A shows evaluation of hematology parameters (mean±s.d., n=3), FIG. 12B shows the biochemistry profile (mean±s.d., n=3), and FIG. 12C shows cytokine panels (mean±s.d., n=3). FIG. 12D shows representative hematoxylin and eosin staining of heart, lung, liver, spleen and kidney for mice 24 hr post intravenous administration of LNP luc mRNA systems containing different ionizable lipids (KC2 or SM102) formulated with different buffers or saline control. Ionizable lipid/DSPC/cholesterol/PEG-DMG (50/10/38.5/1.5 mol %). The values were within the range of reference intervals of normality.

### DETAILED DESCRIPTION

### Methods for Producing LNPs Having Bleb Structures

The method described herein causes the formation of one or bleb in a nanoparticle. As used herein, the term “bleb”, “bleb structure” or “bleb compartment”, means a protrusion formed on a lipid nanoparticle or a space enclosed by a lipid layer in a lipid nanoparticle as observed using microscopy as described herein.

In one embodiment, the method for producing LNPs with bleb structures comprises dissolving lipid components (e.g., ionizable lipid, a sterol, a helper lipid and a polymer-lipid) at appropriate ratios in an organic solvent (e.g., ethanol) or organic solvent/aqueous mixture. An aqueous phase is prepared separately containing the nucleic acid in a buffer. In order to form the lipid nanoparticles, the aqueous phase is combined with the organic solvent-lipid mixture. Lipid nanoparticles form upon mixing and/or thereafter.

Combining the aqueous phase and the organic-solvent-lipid mixture may be carried out in a mixing device (e.g., in-line mixer), such as a T-junction mixer with specialized pumps (e.g., a T-tube mixer), a herringbone micromixer, a toroidal mixer, a multi-inlet vortex mixer or other suitable mixing devices known to those of skill in the art. The aqueous phase and organic-solvent lipid mixture may be introduced to the mixer as two separate respective streams via pumps. In some embodiments, the mixing device refers to a device comprising two or more inlets meeting in a central mixing region and an outlet through which the mixture exits the device. The volumetric flow rate of each stream may be the same or different and the respective flow rates of each stream may be adjusted to achieve optimal mixing and/or LNP formation.

Examples of suitable solvents to prepare the organic solvent-lipid mixture are organic solvents including ethanol, isopropanol, methanol and acetone. In one embodiment, the solvent-lipid mixture comprises ethanol.

In alternate embodiments, the LNPs are prepared by solvent injection. In one embodiment, such method comprises dissolving lipids in an organic solvent and subsequent step-wise dilution of the resultant solution with an aqueous solution (e.g., buffer). This controlled step-wise dilution is achieved by mixing the aqueous and lipid streams together in a container.

The organic solvent used to prepare the LNPs may be substantially removed by dialysis or other suitable solvent removal methods, such as by size exclusion chromatography, dialysis or tangential flow filtration. Size exclusion chromatography may involve passing the LNPs comprising organic solvent over a column that is pre-equilibrated with the buffer solution into which the LNP is to be exchanged. Dialysis is carried out using known techniques and, without being limiting, typically includes the use of a buffer solution that is 200 to 500 times the volume of the sample and one or more buffer exchanges throughout the course of dialysis.

In one embodiment, the solvent removal method eliminates most of the solvent such that less than 3% by weight remains. The residual solvent is quantified by gas chromatography (GC).

In one embodiment, the pH of the buffer-exchanged, external solution of the LNP is between 6 and 8 or between 6.5 and 7.5. Without being bound by theory, the buffer exchange with a buffer having a higher pH may cause bleb formation.

### Lipid Components

As noted, the lipid components used to prepare the LNP include ionizable lipid, at least one of a sterol and a neutral lipid and a polymer-lipid conjugate at appropriate ratios in an organic solvent. Each lipid component is described in more detail below.

### Ionizable Lipids

As used herein, the term “cationic, ionizable lipid” refers to a lipid that, at a given pH, such as physiological pH, is in an electrostatically neutral form and that accepts protons, thereby becoming electrostatically positively charged, and for which the electrostatically neutral form has a calculated logarithm of the partition coefficient between water and 1-octanol (i.e., a c Log P) greater than 8. In some embodiments, the cationic lipid has a pKa that is between 5.0 and 8.0.

As used herein, the term “bleb inducing ionizable lipid” refers to an ionizable lipid that when formulated in a lipid nanoparticle as described in the examples exhibits at least a 10% increase in the number of particles having blebs (in a sample size of at least 200) as compared to a baseline formulation that is otherwise identical and prepared by an identical formulation process but contains SM-102 ionizable lipid when formulated in 300 mM sodium citrate buffer. In one non-limiting example, the bleb inducing ionizable lipid is KC2.

In one embodiment, the ionizable lipid has a protonatable amino head group; at least two lipophilic chains, wherein the protonatable amino head group has a central nitrogen atom or carbon atom to which each of the two lipophilic chains are directly bonded; each lipophilic chain has between 15 and 30 carbon atoms in total; and wherein the ionizable lipid has (i) a pKa of between 6 and 8; and (ii) a log P of at least 11. The two lipophilic chains may be linear or branched and are optionally substituted with heteroatoms. Functional groups containing heteroatoms such as S, O, P and/or N may be introduced into the lipophilic chains in order to make the ionizable lipid more biodegradable in vivo. An example of a biodegradable functional group is an ester. However, as would be appreciated by those of skill in the art, other known biodegradable groups may be included in the lipophilic chains.

In one embodiment, the ionizable lipid has a pKa value that is between about 6.0 and about 8.0 or between about 6.25 and about 7.5.

The mole percentage of the ionizable lipid in the LNP may vary as required and in some embodiments is between 5 and 60 mol % or between 8 and 55 mol % or any value therebetween.

In one non-limiting embodiment, the ionizable amino lipid is selected from a KC2-type lipid or any other suitable ionizable lipid, including those described in co-pending PCT/CA2022/050835 entitled “Method for Producing an Ionizable Lipid”, filed on May 26, 2022 or a sulfur-containing lipid as described in PCT/CA2022/050042 filed on Jan. 12, 2022 entitled “Sulfur-Containing Lipids”, each of which are incorporated herein by reference in their entirety.

The LNP optionally additionally comprises a small amount of a permanently charged lipid(s) (non-ionizable), such as a permanently charged cationic lipid. Such permanently charged cationic lipid may be a lipid having a quarternary amine and includes without limitation N-[1-(2,3-dioleyloxy) propyl]-N,N,N-trimethylammonium chloride (DOTMA), N-[1-(2,3-dioleoyloxy) propyl]-N,N,N-trimethylammonium chloride (DOTAP), Dioctadecylamido-glycylspermine (DOGS), 3b-[N—(N′,N′-dimethylaminoethyl) carbamoyl]cholesterol (DC-Chol) and/or 1,2-dioleoyl-sn-glycero-3-ethylphosphocholine (DOEPC). In one embodiment, the LNP comprises less than 2.5 mol % of the permanently charged lipid.

### Neutral Lipid Components

The lipid nanoparticle (LNP) described herein may comprise a helper lipid. The term “helper lipid” includes any vesicle-forming lipid (e.g., bilayer-forming lipid) that may be selected from a phosphatidylcholine lipid, sphingomyelin, or mixtures thereof. In some embodiments, the helper lipid is selected from sphingomyelin, distearoylphosphatidylcholine (DSPC), dioleoylphosphatidylcholine (DOPC), 1-palmitoyl-2-oleoyl-phosphatidylcholine (POPC) and dipalmitoyl-phosphatidylcholine (DPPC). In certain embodiments, the helper lipid is POPC, DOPC, DSPC or sphingomyelin. In one embodiment, the helper lipid is DSPC. The helper lipid content may include mixtures of two or more different types of different helper lipids.

The phosphatidylcholine content of the lipid nanoparticle in some embodiments is greater than 5 mol %, greater than 10 mol %, 15 mol %, greater than 20 mol %, greater than 25 mol %, greater than 30 mol %, greater than 32 mol %, greater than 34 mol %, greater than 36 mol %, greater than 38 mol %, greater than 40 mol %, greater than 42 mol %, greater than 44 mol %, greater than 46 mol %, greater than 48 mol % or greater than 50 mol %. In some embodiments, the upper limit of phosphatidylcholine content is 70 mol %, 65 mol %, 60 mol %, 55 mol %, 50 mol %, 45 mol %, 40 mol %, 35 mol %, 30 mol %, 25 mol %, 20 mol %, 15 mol %, 10 mol % or 7.5 mol %. The disclosure also encompasses sub-ranges of any combination of the foregoing numerical upper and lower limits.

For example, in certain embodiments, the phosphatidylcholine content is from 5 mol % to 60 mol %, 10 mol % to 60 mol %, 15 mol % to 60 mol %, 20 mol % to 60 mol % or 25 mol % to 60 mol % or 30 mol % to 60 mol % or 35 mol % to 60 mol % or 40 mol % to 60 mol % of total lipid present in the lipid nanoparticle. The phosphatidylcholine lipid content is determined based on the total amount of lipid in the lipid nanoparticle, including the sterol.

### Sterol

The LNP further includes a sterol in some embodiments. The term “sterol” refers to a naturally-occurring or synthetic compound having a gonane skeleton and that has a hydroxyl moiety attached to one of its rings, typically the A-ring.

Examples of sterols include cholesterol, or a cholesterol derivative, the latter referring to a cholesterol molecule having a gonane structure and one or more additional functional groups.

The cholesterol derivative includes β-sitosterol, 3-sitosterol, campesterol, stigmasterol, fucosterol, or stigmastanol, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β[N—(N′N′-dimethylaminoethyl) carbamoyl cholesterol (DC-Chol), 24(S)-hydroxycholesterol, 25-hydroxycholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol or a salt or ester thereof.

In one embodiment, the sterol is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is cholesterol and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is a cholesterol derivative and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In one embodiment, the combined (i) sterol content (e.g., cholesterol or cholesterol derivative thereof); and (ii) neutral lipid content is at least 50 mol %; at least 55 mol %, at least 60 mol %, at least 65 mol %, at least 70 mol %, at least 75 mol %, at least 80 mol % or at least 85 mol % based on the total lipid present in the lipid nanoparticle.

### Hydrophilic Polymer-Lipid Conjugate

In one embodiment, the lipid nanoparticle comprises a hydrophilic-polymer lipid conjugate capable of incorporation into the LNP. The conjugate includes a vesicle-forming lipid having a polar head group, and covalently attached to the head group, a polymer chain that is hydrophilic. In some embodiments, the hydrophilic polymer-lipid conjugate is absent or present at low molar amounts, such as less than about 0.5 mol %.

Examples of hydrophilic polymers include polyethyleneglycol (PEG), polyvinylpyrrolidone, polyvinylmethylether, polyhydroxypropyl methacrylate, polyhydroxypropylmethacrylamide, polyhydroxyethyl acrylate, polymethacrylamide, polydimethylacrylamide, polymethyloxazoline, polyethyloxazoline, polyhydroxyethyloxazoline, polyhydroxypropyloxazoline, polysarcosine and polyaspartamide. In one embodiment, the hydrophilic polymer-lipid conjugate is a PEG-lipid conjugate. The hydrophilic polymer lipid conjugate may also be a naturally-occurring or synthesized oligosaccharide-containing molecule, such as monosialoganglioside (G_{M1}). The ability of a given hydrophilic-polymer lipid conjugate to enhance the circulation longevity of the LNPs herein could be readily determined by those of skill in the art using known methodologies.

The hydrophilic polymer lipid conjugate may be present in the nanoparticle at 0.5 mol % to 5 mol %, or at 0.5 mol % to 3 mol %, or at 0.5 mol % to 2.5 mol % or at 0.5 mol % to 2.0 mol % or at 0.5 mol % to 1.8 mol % of total lipid. In certain embodiments, the hydrophilic polymer lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

In another embodiment, the PEG-lipid conjugate is present in the nanoparticle at 0.5 mol % to 5 mol %, or at 0.5 mol % to 3 mol % or at 0.5 mol % to 2.5 mol % or at 0.5 mol % to 2.0 mol % or at 0.5 mol % to 1.8 mol % of total lipid. In certain embodiments, the PEG-lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

In one embodiment, the hydrophilic polymer-lipid is selected based on its exchangeability from the lipid nanoparticles. Such property may facilitate in vivo efficacy due to at least partial loss of the hydrophilic polymer-lipid conjugate from the LNP as it reaches a target site in vivo.

In such embodiment, the lipid moiety of the hydrophilic polymer-lipid conjugate typically has acyl chain lengths of less than 18 and having 0-2 double bonds in one or both of the acyl chains.

In some embodiments, the hydrophilic polymer-lipid conjugate is a PEG-lipid conjugate selected from dimyristoylphosphatidylethanolamine-PEG (DMPE-PEG), dipalmitoylphosphatidylethanolamine-PEG (DPPE-PEG), dioleylphosphatidylethanolamine-PEG (DOPE-PEG), dipalmitoylphosphatidylethanolamine-PEG (DPPE-PEG), dimyristoyldiglyceride-PEG (DMG-PEG) or cholesterol-PEG (Chol-PEG).

In an alternative embodiment, the LNP comprises an oligosaccharide-lipid conjugate to enhance circulation longevity.

### LNP Morphology

The inventors have found that the bleb structure observed in certain LNP formulations of nucleic acid correlate with in vitro and/or in vivo transfection potency. Further, the inventors have found that such structure can be induced by adjusting the formulation conditions and/or by selection of the ionizable, cationic lipid component.

Adjusting the formulation conditions involves selecting a buffer concentration during formulation that induces blebs. The concentration of the buffer may be greater than 50 mM, 100 mM, 150 mM, 200 mM, 225 mM, 250 mM, 260 mM, 275 mM or 285 mM. In one embodiment, the buffer concentration is between 50 and 350 mM, 75 and 350 mM, 100 and 350 mM, 220 and 350 or 250 and 350 mM.

In one embodiment, the buffer is multivalent. An example of such a buffer is citrate, which has two carboxylic acid moieties. In further embodiments, the buffer may have a valency between 2 and 6. For example, the following compounds 1-7 could serve as buffers:

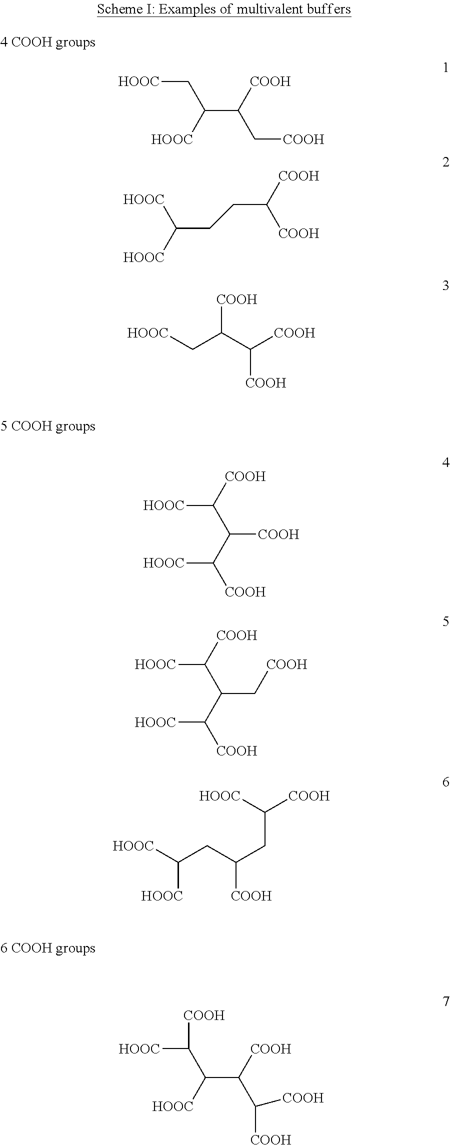

Without being bound by theory, the ability of high concentrations of buffers to induce the formation of bleb structure is likely related to the structure formed on mixing high concentrations of the buffer containing nucleic acid with the lipid solution. The aqueous compartments in these structures would logically contain high levels of the buffer. The pH of the buffer comprising the nucleic acid before mixing with the lipid organic solvent mixture in some embodiments is between 3 and 5, more typically between 3.5 and 4.5. As the pH is raised during dialysis, the interior aqueous compartment(s) would most likely maintain a low pH in comparison to the external environment. The neutral form of the ionizable cationic lipid can readily permeate across lipid bilayers to access these low pH compartments where they would become protonated and trapped. If the buffering capacity of these compartments is sufficient then such a process could eventually strip the ionizable lipid away from the mRNA. The mRNA would then be expected to segregate into an aqueous compartment (water is readily permeable across lipid bilayers) and the structural lipid components (e.g., DSPC/cholesterol) form the inner monolayer.

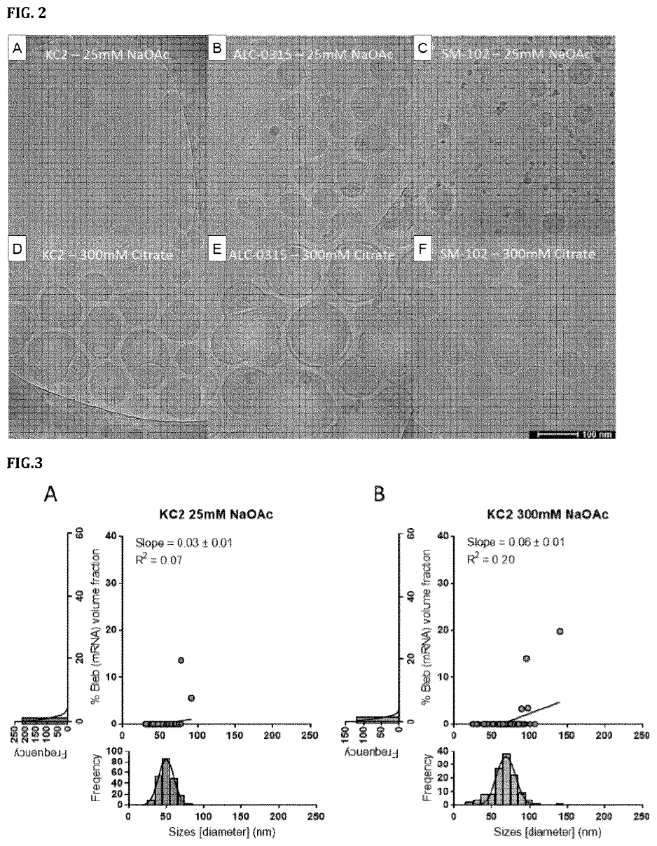

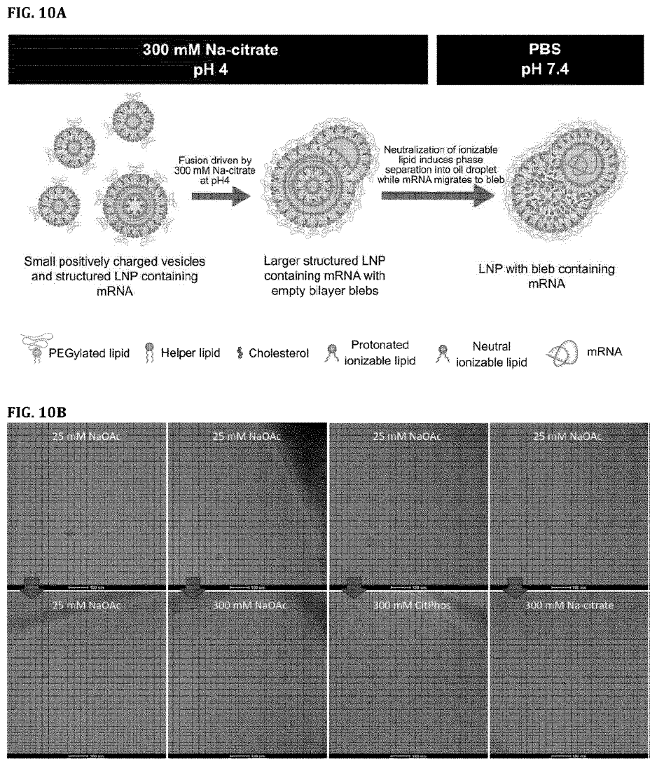

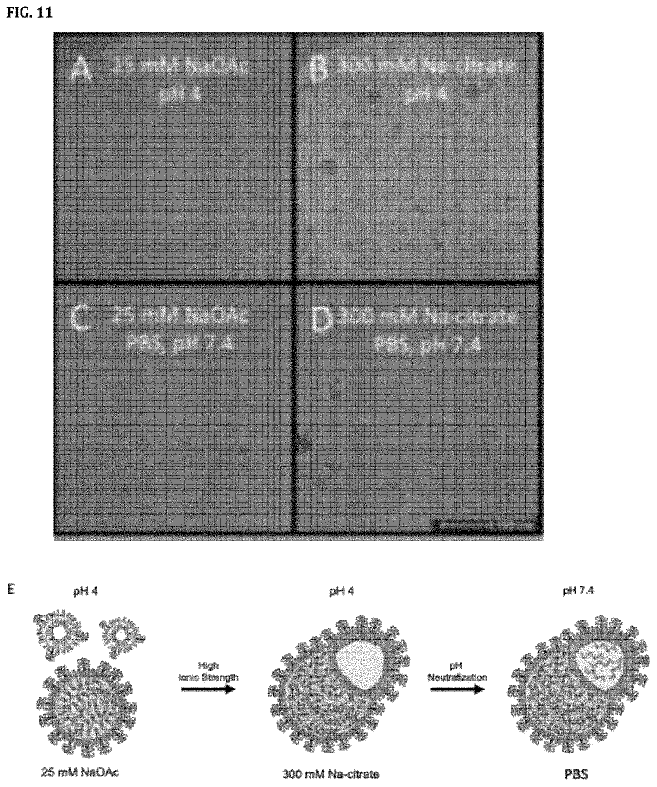

Without being limited by theory, examination of the blebs (see FIG. 2D-F) indicates that the portion of the lipids separating the bleb structure from the external environment have a bilayer structure, suggesting that the interior of the bleb has an aqueous polar character. It is believed that eventually the permeable nature of membranes to protons will prevail, resulting in the accumulated ionizable lipid locating into the neutral, oil form forming the hydrophobic compartment observed by cryo-TEM. A predicted model of the mechanism of bleb formation is depicted in FIG. 10A. Without being limiting, it is believed that citrate and other buffer components would also be expected to migrate to these polar regions.

In one embodiment, the LNP has a hydrophobic compartment disposed between two leaflets of a bilayer. In another embodiment, the bleb compartment is surrounded on one side by an inner leaflet of the lipid layer enclosing the hydrophobic compartment and on another side by a portion of the lipid layer that forms an external bilayer.

The bleb morphology may be correlated with improved transfection potency. In one embodiment, the LNP has a transfection potency in vitro or in vivo that is at least 2-fold greater, 2.5-fold greater, 3.0-fold greater, 3.5-fold greater or 4.0-fold greater than a transfection potency of a baseline lipid nanoparticle that is prepared by mixing lipids in an organic solvent with the nucleic acid in 25 mM sodium acetate at pH 4.0 thereby producing the baseline lipid nanoparticle with a solid core, but that is otherwise identical to the nucleic-acid lipid nanoparticle. The transfection potency is measured as set forth in the Example section.

In another embodiment, the lipid nanoparticle is formulated in a buffer having a concentration of at least 100 mM and the lipid nanoparticle exhibits at least a 10% increase in gene expression of an encapsulated mRNA as compared to a baseline formulation that is otherwise identical but contains 25 mM sodium acetate buffer.

The copy number of cargo encapsulated in the lipid nanoparticle may also correlate with bleb morphology. In one embodiment, the mRNA copy number/LNP is 2-10 or 4-8. The copy number of cargo molecules may also correlate with the nanoparticle size. In one embodiment, the nanoparticle has a diameter of between 60 and 110 nm.

In another embodiment, the copy number of mRNA cargo correlates with cross-sectional area of the bleb. In one embodiment, the bleb has a minimum cross-sectional area of 150 nm², or 180 nm² or 200 nm² to accommodate more cargo (e.g., mRNA) as calculated from a cryoEM image as described in the Example section herein.

### Nucleic Acid Cargo

The cargo includes any type of nucleic acid that can be incorporated into an LNP using the method described herein. Without intending to be limiting, the cargo may be a therapeutic agent, a prodrug or a diagnostic agent. In one embodiment, the cargo is a nucleic acid, peptide-nucleic acid complex or a protein-nucleic acid complex.

The nucleic acid includes, without limitation, RNA, including small interfering RNA (siRNA), small nuclear RNA (snRNA), micro RNA (miRNA), messenger RNA (mRNA) or DNA such as vector DNA or linear DNA. The nucleic acid length can vary and can include nucleic acid of 1-50,000 nucleotides in length. The nucleic acid can be in any form, including single stranded DNA or RNA, double stranded DNA or RNA, or hybrids thereof. Single stranded nucleic acid includes antisense oligonucleotides. The nucleic acid may be conjugated to another molecule, including a targeting moiety. An example of such a nucleic acid conjugate is an antibody-nucleic acid conjugate, or an oligosaccharide-nucleic acid conjugate, such as a GalNAc-nucleic acid conjugate.

In one embodiment, the cargo is an mRNA, which includes a polynucleotide that encodes at least one peptide, polypeptide or protein. The mRNA includes, but is not limited to, small activating RNA (saRNA) and trans-amplifying RNA (taRNA).

The mRNA as used herein encompasses both modified and unmodified mRNA. In one embodiment, the mRNA comprises one or more coding and non-coding regions. The mRNA can be purified from natural sources, produced using recombinant expression systems and optionally purified, or may be chemically synthesized.

In those embodiments in which an mRNA is a chemically synthesized molecule, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNAs of the disclosure may be synthesized according to any of a variety of known methods. For example, mRNAs in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before encapsulation to remove undesirable impurities including various enzymes and other reagents used during mRNA synthesis.

The present disclosure may be used to encapsulate mRNAs of a variety of lengths. In some embodiments, the present disclosure may be used to encapsulate in vitro synthesized mRNA ranging from about 1-20 kb, about 1-20 kb, about 1-15 kb, about 1-10 kb, about 5-20 kb, about 5-15 kb, about 5-12 kb, about 5-10 kb, about 8-20 kb, or about 8-15 kb in length.

Typically, mRNA synthesis includes the addition of a “cap” on the 5′ end, and a “tail” on the 3′ end. The presence of the cap is advantageous in that it may provide resistance to nucleases found in eukaryotic cells. The presence of a “tail” serves to protect the mRNA from exonuclease degradation.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 1 and 500 nucleotides in length or 50 and 500 nucleotides in length or longer.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 1 and 500 nucleotides in length or 50 and 500 nucleotides in length or longer.

While mRNA provided from in vitro transcription reactions may be desirable in certain embodiments, other sources of mRNA are contemplated, such as mRNA produced from bacteria, fungi, plants, and/or animals.

The mRNA sequence may comprise a reporter gene sequence, although the inclusion of a reporter gene sequence in pharmaceutical formulations for administration is optional. Such sequences may be incorporated into mRNA for in vitro studies or for in vivo studies in animal models to assess expression and biodistribution.

In another embodiment, the cargo is an siRNA. An siRNA becomes incorporated into endogenous cellular machineries to result in mRNA breakdown, thereby preventing transcription. Since RNA is easily degraded, its incorporation into a delivery vehicle can reduce or prevent such degradation, thereby facilitating delivery to a target site.

The siRNA encompassed by embodiments of the disclosure may be used to specifically inhibit expression of a wide variety of target polynucleotides. The siRNA molecules targeting specific polynucleotides for any therapeutic, prophylactic or diagnostic application may be readily prepared according to procedures known in the art. An siRNA target site may be selected and corresponding siRNAs may be chemically synthesized, created by in vitro transcription, or expressed from a vector or PCR product. A wide variety of different siRNA molecules may be used to target a specific gene or transcript. The siRNA may be double-stranded RNA, or a hybrid molecule comprising both RNA and DNA, e.g., one RNA strand and one DNA strand. The siRNA may be of a variety of lengths, such as 1 to 30 nucleotides in length or 15 to 30 nucleotides in length or 20 to 25 nucleotides in length. In certain embodiments, the siRNA is double-stranded and has 3′ overhangs or 5′ overhangs. In certain embodiments, the overhangs are UU or dTdT 3′. In particular embodiments, the siRNA comprises a stem loop structure.

In a further embodiment, the cargo molecule is a microRNA or small nuclear RNA. Micro RNAs (miRNAs) are short, noncoding RNA molecules that are transcribed from genomic DNA, but are not translated into protein. These RNA molecules are believed to play a role in regulation of gene expression by binding to regions of target mRNA. Binding of miRNA to target mRNA may downregulate gene expression, such as by inducing translational repression, deadenylation or degradation of target mRNA. Small nuclear RNA (snRNA) are typically longer noncoding RNA molecules that are involved in gene splicing. The snRNA molecules may have therapeutic or diagnostic importance in diseases that are an outcome of splicing defects.

In another embodiment, the cargo is a DNA vector. The encapsulated DNA vectors may be administered to a subject for the purpose of repairing, enhancing or blocking or reducing the expression of a cellular protein or peptide. In another embodiment, the encapsulated DNA vectors may be administered to a subject for diagnosis of disease. The DNA vector may localize in target cells (e.g., rapidly dividing cells) and expression of encoded DNA may be used to provide a measurable signal. Accordingly, the nucleotide polymers can be nucleotide sequences including genomic DNA, cDNA, or RNA.

As will be appreciated by those of skill in the art, the vectors may encode promoter regions, operator regions or structural regions. The DNA vectors may contain double-stranded DNA or may be composed of a DNA-RNA hybrid. Non-limiting examples of double-stranded DNA include structural genes, genes including operator control and termination regions, and self-replicating systems such as vector DNA.

Single-stranded nucleic acids include antisense oligonucleotides (complementary to DNA and RNA), ribozymes and triplex-forming oligonucleotides. In order to have prolonged activity, the single-stranded nucleic acids will most advantageously have some or all of the nucleotide linkages substituted with stable, non-phosphodiester linkages, including, for example, phosphorothioate, phosphorodithioate, phophoroselenate, or O-alkyl phosphotriester linkages.

The DNA vectors may include nucleic acids in which modifications have been made in one or more sugar moieties and/or in one or more of the pyrimidine or purine bases. Such sugar modifications may include replacement of one or more hydroxyl groups with halogens, alkyl groups, amines, azido groups or functionalized as ethers or esters. In another embodiment, the entire sugar may be replaced with sterically and electronically similar structures, including aza-sugars and carbocyclic sugar analogs. Modifications in the purine or pyrimidine base moiety include, for example, alkylated purines and pyrimidines, acylated purines or pyrimidines, or other heterocyclic substitutes known to those of skill in the art.

The DNA vector may be modified in certain embodiments with a modifier molecule such as a peptide, protein, steroid or sugar moiety. Modification of a DNA vector with such molecule may facilitate delivery to a target site of interest. In some embodiments, such modification translocates the DNA vector across a nucleus of a target cell. By way of example, a modifier may be able to bind to a specific part of the DNA vector (typically not encoding of the gene-of-interest), but also has a peptide or other modifier that has nucleus-homing effects, such as a nuclear localization signal. A non-limiting example of a modifier is a steroid-peptide nucleic acid conjugate as described by Rebuffat et al., 2002, Faseb J. 16 (11): 1426-8, which is incorporated herein by reference. The DNA vector may contain sequences encoding different proteins or peptides. Promoter, enhancer, stress or chemically-regulated promoters, antibiotic-sensitive or nutrient-sensitive regions, as well as therapeutic protein encoding sequences, may be included as required. Non-encoding sequences may be present as well in the DNA vector.

The nucleic acids used in the present disclosure can be isolated from natural sources, obtained from such sources as ATCC or GenBank libraries or prepared by synthetic methods. Synthetic nucleic acids can be prepared by a variety of solution or solid phase methods. Generally, solid phase synthesis is preferred. Known procedures for solid phase synthesis of nucleic acids by phosphite-triester, phosphotriester, and H-phosphonate chemistries are widely available.

In one embodiment, the DNA vector is double stranded DNA and comprises more than 700 base pairs, more than 800 base pairs or more than 900 base pairs or more than 1000 base pairs.

In another embodiment, the cargo includes a nucleic acid that is part of a peptide- or protein-nucleic acid complex that is used for nucleic acid editing. In one embodiment, the peptide, protein, peptide-nucleic acid complex or the protein-nucleic acid complex is charged. In one embodiment, the peptide, protein, peptide-nucleic acid complex or the protein-nucleic acid complex is negatively charged.

Examples of gene editing systems that can be incorporated into an LNP include a CRISPR, TALEN and zinc finger nuclease gene editing system.

The cargo most advantageously comprises Class II Cas nuclease families of proteins, which are enzymes with DNA endonuclease activity, and can be directed to cleave a desired nucleic acid target by an appropriate guide RNA. The enzyme and guide RNA form a complex referred to as a ribonucleoprotein (RNP). In some embodiments, the nuclease is a Class II CRISPR enzyme, which is further subdivided into Types II, V and VI. According to one embodiment, the Cas protein is part of a Type II CRISPR/Cas system, such as a Cas9 protein or a Cpf1 protein.

In another embodiment, the Cas protein is part of a Type V CRISPR/Cas system, such as Cas12a. In another embodiment, the Cas protein is a Cas 13a, which is an RNA endonuclease and cleaves single-stranded RNA.

The guide RNA can direct the Cas nuclease to the target sequence on a target nucleic acid molecule, where the guide RNA hybridizes to the target sequence and the Cas nuclease cleaves or modulates the sequence. In some embodiments, the guide RNA binds to a class 2 nuclease, thereby providing specificity of cleavage.

Guide RNAs for the CRISPR/Cas9 nuclease system include CRISPR RNA (crRNA) or tracr RNA (tracr). In some embodiments, the crRNA can include a targeting sequence that is complementary to and hybridizes to a target sequence on a target nucleic acid molecule. The crRNA can also include a flagpole that is complementary to, and hybridize to, a portion of tracrRNA. In some embodiments, the crRNA can correspond to the structure of a naturally-occurring crRNA transcribed from a bacterial CRISPR locus, wherein the targeting sequence acts as a spacer for the CRISPR/Cas9 system. The flagpole corresponds to the part of the repetitive sequence adjacent to the spacer above the CRISPR locus.

The guide RNA of the RNP can target any sequence of interest through the targeting sequence of crRNA. In some embodiments, the targeting sequence of the guide RNA and the target sequence on the target nucleic acid molecule may be 100% complementary. In other embodiments, the targeting sequence of the guide RNA and the target sequence on the target nucleic acid molecule can comprise at least one mismatch.

The length of the targeting sequence may depend on the RNP system and components used. For example, different Cas proteins from different bacterial species have various optimal targeting sequence lengths. In some embodiments, the targeting sequence can comprise a length of 18 to 24 nucleotides. In some embodiments, the targeting sequence can comprise 19-21 nucleotides in length. In some embodiments, the targeting sequence can comprise a length of 20 nucleotides.

Other protein or peptide complexes besides protein or peptide-nucleic acid complexes are encompassed by the disclosure.

While protein or peptide-nucleic acid complexes are described above, the cargo may also be a protein or peptide that is charged below the pKa of the LNP. Non-limiting examples of peptides or proteins that may be incorporated into the LNP include peptides or proteins that are charged at a pH of interest.

### Clinical and Non-Clinical Uses of the LNP Herein

In some embodiments, the nucleic acid-LNP is part of a pharmaceutical composition and is administered to treat and/or prevent a disease condition. The treatment may provide a prophylactic (preventative), ameliorative or a therapeutic benefit. The pharmaceutical composition will be administered at any suitable dosage.

The nucleic acid-LNPs described herein may be used to treat and/or prevent any disease, disorder or condition in a mammalian subject. This includes a disease, disorder or condition, such as cancer, infectious diseases such as bacterial, viral, fungal or parasitic infections, inflammatory and/or autoimmune disorders, including treatments that induce immune tolerance and cardiovascular diseases such as hypertension, cardiac arrhythmia and restenosis.

Examples of cancers include lung cancer, colon cancer, rectal cancer, anal cancer, bile duct cancer, small intestine cancer, stomach (gastric) cancer, esophageal cancer; gallbladder cancer, liver cancer, pancreatic cancer, appendix cancer, breast cancer, ovarian cancer; cervical cancer, prostate cancer, renal cancer (e.g., renal cell carcinoma), cancer of the central nervous system, glioblastoma, skin cancer, lymphomas, choriocarcinomas, head and neck cancers, osteogenic sarcomas, and blood cancers. Non-limiting examples of specific types of liver cancer include hepatocellular carcinoma (HCC), secondary liver cancer (e.g., caused by metastasis of some other non-liver cancer cell type), and hepatoblastoma.

Non-limiting examples of other diseases, disorders or conditions that may be treated by the nucleic acid-LNPs herein and that may be attributed at least in part to an immunological disorder include colitis, Crohn's disease, allergic encephalitis, allograft transplant/graft vs. host disease (GVHD), diabetes and multiple sclerosis.

The LNPs herein may also be used in other applications besides the treatment and/or prevention of a disease or disorder. The LNPs may be used to treat conditions such as aging, preventative medicine and/or as part of a personalized medicine regime. In further embodiments, the LNP is used in a diagnostic application.

In one embodiment, the LNP is part of a pharmaceutical composition administered parenterally, i.e., intra-arterially, intravenously, subcutaneously or intramuscularly. In yet a further embodiment, the pharmaceutical compositions are for intra-tumoral administration. In another embodiment, the pharmaceutical compositions are administered intranasally, intravitreally, subretinally, intrathecally or via other local routes. In some embodiments, the nucleic acid-LNP is applied or administered to the skin.

The pharmaceutical composition comprises pharmaceutically acceptable salts and/or excipients. As used herein, the term “pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

As used herein, the term “excipient” means the substances used to formulate active pharmaceutical ingredients (API) into pharmaceutical formulations. Non-limiting examples include mannitol, Captisol®, lactose, starch, magnesium stearate, sodium saccharine, talcum, cellulose, sodium crosscarmellose, glucose, gelatin, sucrose, magnesium carbonate, and the like. Acceptable excipients are non-toxic and may be any solid, liquid, semi-solid excipient that is generally available to those of skill in the art.

The compositions described herein may be administered to a subject. The term subject as used herein includes a human or a non-human subject, including a mammal. The nucleic acid-LNP may be administered as part of a preventative treatment and so the subject is not limited to a patient.

### EXAMPLES

### Material and Methods

The lipid 1,2-distearoyl-sn-glycero-3-phosphocholine (DSPC) and (R)-2,3-Bis(tetradecyloxy) propyl-1-(methoxy polyethylene glycol 2000) carbamate (PEG-DMG) were purchased from Avanti Polar Lipids. The ionizable lipid DLin-KC2-DMA (KC2) was purchased through Dr. Marco Ciufolini, while the ionizable lipids ALC-0315 and SM-102 were purchased from Cayman Chemicals™. Cholesterol was purchased from Sigma Aldrich™. RPMI 1640 and DMEM media was purchased from Thermo Fisher Scientific™. All other chemicals were purchased from Sigma Aldrich™ unless otherwise stated. siRNA against firefly luciferase was purchased from Integrated DNA Technologies (IDT), with the following sequence: Sense: cuuAcGcuGAGuAcuucGAdTsdT, Antisense: UCGAAGuACUcAGCGuAAGdTsdT. The lower-case letters denote 2′OMe modifications, while the phosphorothioate linkages are denoted by the lower case “s” between the 3′-deoxythymidine (dT) overhangs. ARCA Capped™ Firefly Luciferase mRNA (5mCTP, ψUTP) was purchased from APExBIO.

### LNP Formulation and Characterization

LNPs were formulated as previously described, with slight modifications. All formulations were initially formulated by first dissolving lipids (KC2, DSPC, cholesterol PEG-DMG) into ethanol to a final concentration of 10 mM and at set ratios. The lipid mixture was then rapidly mixed with an aqueous solution of 25 mM sodium acetate/300 mM sodium acetate/300 mM Na-citrate phosphate/300 mM sodium Na-citrate at pH 4 using a T-junction mixer at a 1:3 ratio and a final flow rate of 20 mL min-1. The resulting mixture was dialyzed 500-fold in either 25 mM sodium acetate, 25 mM Na-citrate phosphate, 300 mM sodium acetate, 300 mM Na-citrate phosphate, 300 mM sodium Na-citrate, or PBS. The LNPs were sterile-filtered with a 0.2 μm stupor membrane and concentrated in 10K Amicon™ ultracentrifugation units. Total lipid content was determined by extrapolation using the Cholesterol E Kit™ (Wako Diagnostics™, Mountain View, CA). Particle size was measured as the number mean average, determined via dynamic light scattering using the Malvern Zetasizer Nano ZS™.

For siRNA/mRNA in vitro studies: LNPs were formulated as described, but with the inclusion of 0.1 mol % of the lipophilic tracers DiI and siRNA against luciferase (siLuc) or mRNA luciferase (mLuc) in the aqueous solution at an amine-to-phosphate (N/P) ratio of three (siRNA) or six (mRNA). The encapsulation efficiency of the siRNA was quantified using the Quant-iT™ RiboGreen™ RNA Assay Kit (Wako Diagnostics™, Mountain View, CA).

For in vivo bioluminescence imaging studies: LNPs were formulated as described, but with the inclusion of 0.5 mol % of the lipophilic tracers DiD in the initial lipid mixture.

### Cryo-Transmission Electron Microscopy (Cryo-TEM)

The freshly prepared LNPs were concentrated (20-25 mg/mL of total lipid) and added to glow-discharged copper grids (3-5 μL), and plunge-frozen using an FEI Mark IV Vitrobot™ (FEI, Hillsboro, OR) to generate vitreous ice. Grids were moved into a Gatan 70° cryo-tilt transfer system pre-equilibrated to at least −180° C. and then inserted into the microscope. An FEI LaB6 G2 TEM (FEI, Hillsboro, OR) operating at 200 kV under low-dose conditions was used to image all samples with an FEI Eagle 4 K CCD camera. All samples (unless otherwise stated) were imaged at 55000× magnification with a nominal under-focus of 1-2 μm to enhance contrast. All sample preparation and imaging were performed by the UBC Bioimaging Facility (Vancouver, BC).

### Manual LNP Annotation of Cyro-TEM Images

For each formulation, >10 frame of images were selected for annotation. Each of these images contained between 1 to 93 LNPs in the fields of view. Each LNP was analysed for its features related to the bleb structure and the number of nanoparticles relative was counted with either bleb or no bleb. The annotations were then immediately visible to the user as a colour-coded number. An example can be found in the supplementary documents.

For the LNP size [diameter, nm] and % bleb (mRNA) volume fraction, a custom set of FIJI parameter were created for annotation process. LNP size are measured based on the longest diameter of each individual particles. The d_{bleb} (diameter of blebs) are measured based on the number of bleb and the distance from the edge to the mottled mass dense ends. While d_{LNP} (diameter of LNP) is the sum of all diameters of blebs and the electron dense core. The researcher would identify a bleb, click on it and press a shortcut key to create a diameter annotation that identified the feature using the FIJI software. Each LNP are marked with a number to keep track between each frame and count. Average LNP sizes were obtained from the measurement of a minimum of 200 LNP over all representative fields of view.

$$\% bleb ( mRNA ) volume fraction = \frac{\sum 4 / 3 {π ( \frac{{d}_{b l e b}}{2} )}^{3}}{4 / 3 {π ( \frac{{d}_{L N P}}{2} )}^{3}}$$

For the number of copies of mRNA per LNP calculation, we assumed that a single mRNA in a bleb exists in a condensed, spherical form with a volume calculated assuming an mRNA diameter of 2 nm and a length per nucleotide of 0.33 nm¹⁹. For a 2 kb mRNA the radius (r) of the mRNA sphere can be calculated to be approximately 8 nm and the cross-sectional area (πr2) ˜200 nm². The number of copies of mRNA encapsulated per LNP can then be determined based on the average cross-sectional area of the blebs on in the cryo-TEM micrographs divided by the area per monomer.

### Cell Culture and Treatments

A luciferase-expressing human prostate cancer cell line (22Rv1-luc) was cultured and maintained in RPMI 1640 media supplemented with 10% fetal bovine serum (FBS), while the liver carcinoma cell line, HuH7, was cultured in DMEM media supplemented with 10% FBS. For transfecting with LNPs, 13,000 cells per well were seeded into 96-well microplates for 24 hours before treating with the LNPs at siRNA or mRNA doses from 0-10 μg/mL of LNP-siRNA or LNP-mRNA. Following 24 hours of incubation, the media was removed, and cells were lysed with 100 μL of Glo Lysis buffer (Promega™, Wisconsin, US). The lysates were mixed at a 1:1 ratio with luciferase substrate (Promega™, Wisconsin, US) and luminescence was quantified using the CentroXS3 LB 960 luminometer (Berthold Technologies, Germany) and a Spark® Multimode Microplate reader (Tecan, Zürich, Switzerland). Luminescence readings were then normalized to the total protein content per well as determined using the Pierce™ BCA Protein Assay Kit (ThermoFisher™), and transfection was calculated by normalizing to the luminescence values for untreated cells. For cell viability, cells were treated at from 0-10 μg/mL of LNP-mRNA and incubated for 24 h in a 96-well microplate, after which the medium was replaced with that containing 50 μg/mL alamarBlue™ in fresh media (Invitrogen). Cells were incubated for 1.5 h, and the absorbance at 570 nm and 600 nm was then collected on the Spark® Multimode Microplate reader (Tecan, Zürich, Switzerland). The percent cell viability for each experiment was determined by normalizing the averaged blank-corrected absorbance values of four replicate treatment wells against blank-corrected absorbance values of wells administered media alone.

### Bioluminescence Imaging of LNP-mRNA Luciferase Transfection

Female CD-1 mice were individually weighed and imaged prior to intravenous (i.v.) injection of LNPs (0.5 mg/kg mRNA) via tail vein injection. At 3-, 6- and 24 hours post-injection mice were administered D-luciferin substrate solution (150 mg/kg) intraperitoneally 10 min before bioluminescence imaging. Mice belonging to the same dosing groups (n=3) were anesthetized (isoflurane) and placed on the imaging platform in a supine position while being maintained on isoflurane via a nose cone. Mice were imaged 10 min post administration of D-luciferin and imaged on the Xenogen™ IVIS Spectrum In Vivo Imaging System, with an exposure time of 10 sec.

Immediately following the last imaging time point (24 h) of each group of mice, the mice in that group were euthanized, and the liver, spleen, heart, kidney, lungs, adrenals, brain, skin, fat, muscle and bone were collected and imaged ex vivo. For in vivo imaging, bioluminescence values were quantified by measuring the radiance (photons/second/cm²/steradian) in the whole animal, and for focal regions of interest (abdominal region) using the Living Image® software program. For ex vivo imaging, bioluminescence values were also quantified by the radiance (photons/second/cm²/steradian) for each excised organ.

LNP-mRNA Stability, mRNA Integrity and Wheat Germ Extract Assay

LNPs-containing mLuc mRNA and KC2 (10 μg/mL) were incubated in 50% FBS at 0, 3, 6 and 24 h at 37° C. Non-encapsulated mRNA was used as a control incubated in either PBS or 50% FBS under the same conditions. mRNA was extracted from the LNP-mRNA formulations and 50% FBS solution following manufacturer's instructions using a PureLink™ RNA Mini Kit (Thermo Fisher Scientific™). The concentration of the extracted RNAs were measured via a NanoDrop™ by absorption at 280 nm. The extracted mRNAs were loaded into an RNA Chip kit and mRNA integrity was determined by automated electrophoresis using the Agilent 2100 Bioanalyzer. The gel and corresponding electropherograms of the mRNA samples generated through the Agilent 2100 Expert Software™ (Agilent Technologies™) was used to determine mRNA quality in the formulation following incubation in PBS or serum. The extracted mRNA samples (100 ng) were then incubated in wheat germ extract (Promega™, Madison, WI, USA) containing 8U Ribolock inhibitor (Thermo FisherScientific™, Mississauga, ON, Canada), 0.4 μL amino acid mix minus methionine (1M) (Promega, Madison, WI, USA) and 0.4 μL amino acid mix minus cysteine (1 M) (Promega™, Madison, WI, USA) at 25° C. for 1 hour. Luciferase activity was measured using a luminometer (Turner Designs™ TD-20/20).

### Acute Toxicity Studies

To measure the toxicity of the formulations used in vivo, mice (n=3) for 4 groups (KC2-25 mM NaOAc, KC2-300 mM Na-citrate, SM-102-25 mM NaOAc, SM-102-300 mM Na-citrate) were administered i.v. with 1.5 mg/kg LNP luc mRNA formulated based on Onpattro composition and 1 group with saline as control. At 24 hours post i.v. administration, 1 mL of blood was withdrawn through cardiac puncture. Blood was transferred to MiniCollect® K2EDTA and VACUETTER Z Serum Clot Activator Tube with Gel Separator respectively (Fisher Scientific, Ottawa, ON, Canada), and transferred to UBC Centre for Comparative Medicine for further processing, the blood samples for biochemistry panel were allowed to clot at room temp for 30 min and centrifuged at 2000 rpm for 10 min. Serum was then transferred to Eppendrof tubes and kept at 4 degrees. Custom hematology test, biochemistry tests and Mouse Cytokine Multiplex Panel were performed by IDEXX BioAnalytics, Delta, BC, Canada and IDEXX BioAnalytics, MO, USA. Major organs (heart, lungs, liver, spleen and kidney) were also excised at 24 hr post administration for histopathological analysis through hematoxylin and eosin staining by Wax-it Histology Services Inc (Vancouver, BC, Canada).

### Example 1: Nucleic Acid-LNP Systems Prepared in the Presence of High Concentrations of Buffers Exhibit Unusual “Bleb” Structures

The LNP mRNA formulation process involves an initial rapid mixing step of lipid in ethanol with nucleic acid dissolved in a buffer pH 4, usually 25 mM NaOAc. In order to determine the impact of formulating with different buffers at high buffer concentrations LNP were formulated from a composition of KC2/DSPC/cholesterol/PEG-DMG (50/10/38.5/1.5 mol/mol) lipid mixture in ethanol and pH 4 buffers containing luciferase mRNA and either 25 mM NaOAc, 300 mM NaOAc, 300 mM Na-citrate phosphate, or 300 mM Na-citrate using the T-tube procedure detailed in Methods. As shown in Table 1 (Example 2), encapsulation efficiencies greater than 90% in the presence of 300 mM buffer concentrations were achieved for LNP containing the ionizable lipid, KC2, in all buffers.

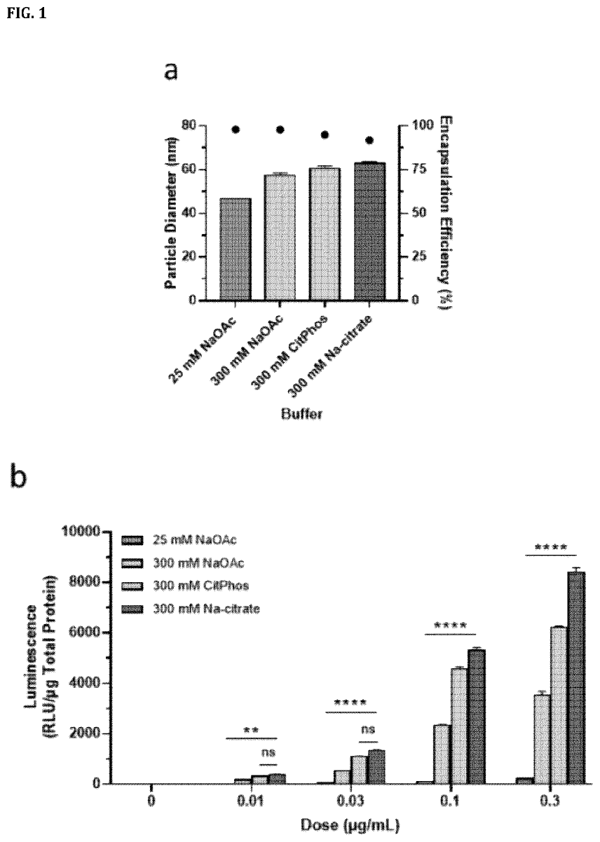

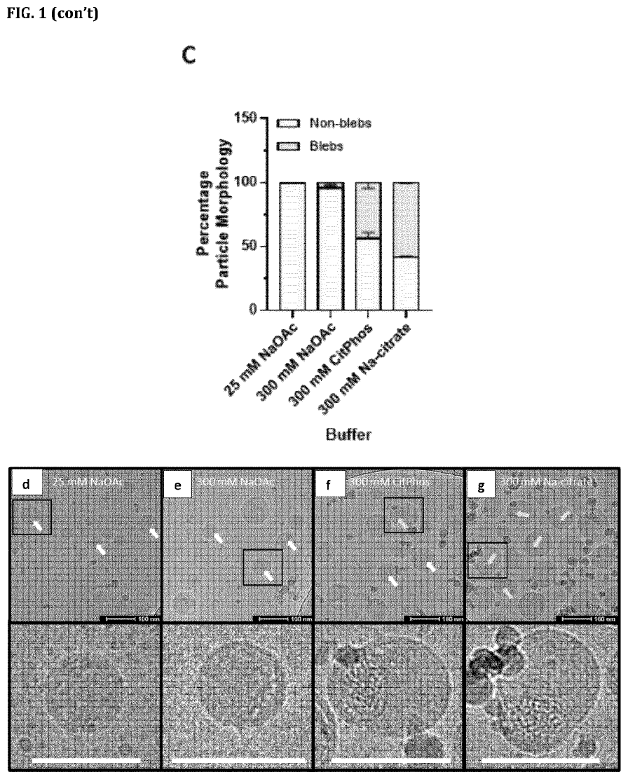

In order to characterize the influence of formulating LNP mRNA systems in a variety of 300 mM buffers (pH 4) on LNP morphology, the structural properties of LNP containing KC2 were investigated using cryo-TEM. As shown in FIG. 1D, the LNP prepared in the presence of 25 mM NAOAc displayed typical “solid core” morphology (FIG. 1D, white arrows) as observed previously. However, increasing the NaOAc ionic strength to 300 mM resulted in a fraction of particles that exhibit unusual “bleb” structures. Interestingly, LNP mRNA formulations prepared in 300 mM NaOAc, 300 mM Na-citrate phosphate or 300 mM Na-citrate led to an increasing proportion of particles that exhibit such bleb morphology (FIG. 1E-G, grey arrows).

High concentrations of pH 4 buffer gave rise to a size increase from ˜45 nm to ˜60 nm diameter (FIG. 1A). KC2 LNP mRNA systems prepared in 300 mM NaOAc, 300 mM CitPhos or 300 mM Na-citrate displayed 2%, 43% and 59% bleb structure respectively (FIG. 1C). Dose titration transfection results with the various buffers (25 mM NaOAc, 300 mM NaOAc, 300 mM citrate and 300 mM Na-citrate; from left to right) are shown in FIG. 1B. The sodium citrate buffer at 300 mM exhibited the highest transfection as measured by luminescence at all doses (0.01-0.3 μg/mL). As further shown in FIG. 1B, increasing the ionic strength of the NaOAc buffer from 25 mM to 300 mM resulted in a remarkable 32-fold increase in luminescence at an LNP mRNA concentration of 0.3 μg mRNA/mL.

### Example 2: The Prevalence of Bleb Structures Depends on the Ionizable Lipid Species as Well as the Concentration of Buffer

In order to determine the influence of different ionizable lipids on the formulation and morphological properties of LNP mRNA systems prepared in high concentrations of pH 4 buffer, LNP luc mRNA systems were formulated with KC2, ALC-0315 and SM-102 as the ionizable lipids and using the 300 mM pH 4 Na-citrate buffer. As noted in Table 1, the resulting systems containing KC2 and SM-102 exhibited excellent encapsulation in the presence of 300 mM Na-citrate buffer and minimal increase in size.

TABLE 1
Mean particle diameter, polydispersity index (PDI) and encapsulation efficiencies of LNP

| | | | Mean Particle | | |
|---|---|---|---|---|---|
| LNP Formulation | | | Diameter ± SEM | | |
| Ionizable Lipid | Nucleic Acid | Buffer | (nm) | Mean PDI ± SEM | Encapsulation (%) |
| KC2 | mRNA | 25 mM NaOAc | 46.65 ± 0.60 | 0.050 ± 0.005 | 98.03 |
| KC2 | mRNA | 300 mM NaOAc | 57.52 ± 1.08 | 0.036 ± 0.015 | 97.99 |
| KC2 | mRNA | 300 mM CitPhos | 60.57 ± 0.86 | 0.042 ± 0.010 | 94.97 |
| KC2 | mRNA | 300 mM Citrate | 62.81 ± 0.94 | 0.053 ± 0.003 | 91.91 |
| ALC-0315 | mRNA | 25 mM NaOAc | 46.61 ± 1.13 | 0.059 ± 0.012 | 84.10 |
| ALC-0315 | mRNA | 300 mM Citrate | 131.87 ± 1.92 | 0.089 ± 0.015 | 72.07 |
| SM-102 | mRNA | 25 mM NaOAc | 45.30 ± 0.67 | 0.093 ± 0.004 | 95.26 |
| SM-102 | mRNA | 300 mM Citrate | 51.33 ± 0.68 | 0.093 ± 0.003 | 95.26 |
| KC2 | SiRNA | 25 mM NaOAc | 51.58 ± 1.01 | 0.068 ± 0.005 | 94.17 |
| KC2 | SiRNA | 300 mM NaOAc | 38.55 ± 1.02 | 0.180 ± 0.002 | 96.09 |
| KC2 | SiRNA | 300 mM CitPhos | 39.51 ± 0.48 | 0.149 ± 0.006 | 96.45 |
| KC2 | SiRNA | 300 mM Citrate | 45.31 ± 0.86 | 0.072 ± 0.008 | 93.23 |

While LNP containing ALC-0315 had lower encapsulation efficiencies for the 300 mM Na-citrate buffer accompanied by large size increases, the most interesting feature concerned the morphologies observed by cryo-TEM. As shown in FIG. 2, in all cases a marked increase in bleb structure was observed using 300 mM citrate. This change was most notable for the KC2 LNP mRNA formulation (FIGS. 2A and 2D). LNP containing SM-102 exhibited low levels (4%) of bleb structures when formulated using the 25 mM NaOAc buffer and higher levels (21%) when prepared using 300 mM Na-citrate (FIGS. 2C and 2F). However, in the case of the LNPs prepared from ALC-0315, appreciable bleb structure (54%) was observed even for the LNPs prepared using the 25 mM NaOAc pH 4 buffer (FIG. 2C).

### Example 3: Increasing the Formation of Bleb Containing mRNA with Higher Concentration Buffers Increases the Sizes of LNP and the Number of mRNA Each Bleb Contains

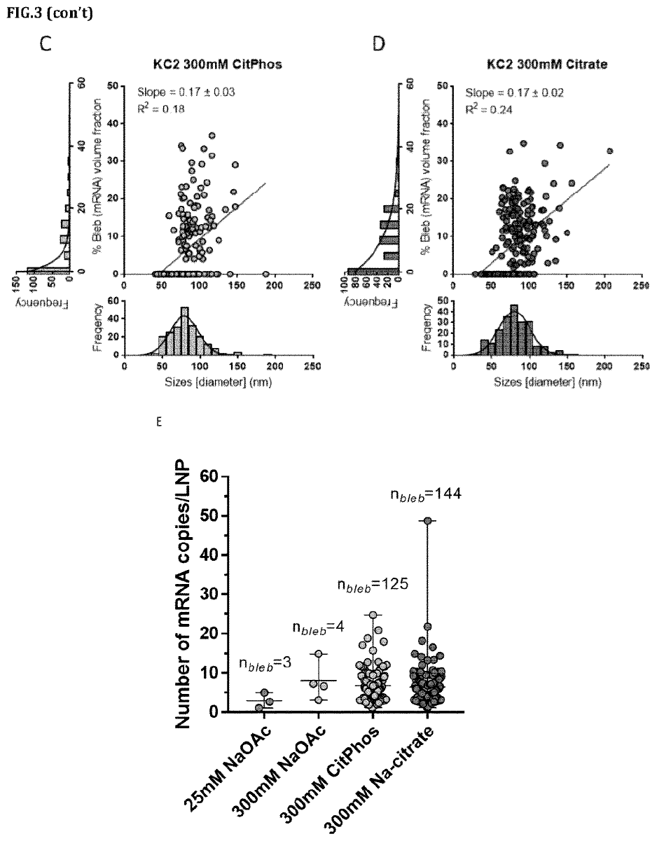

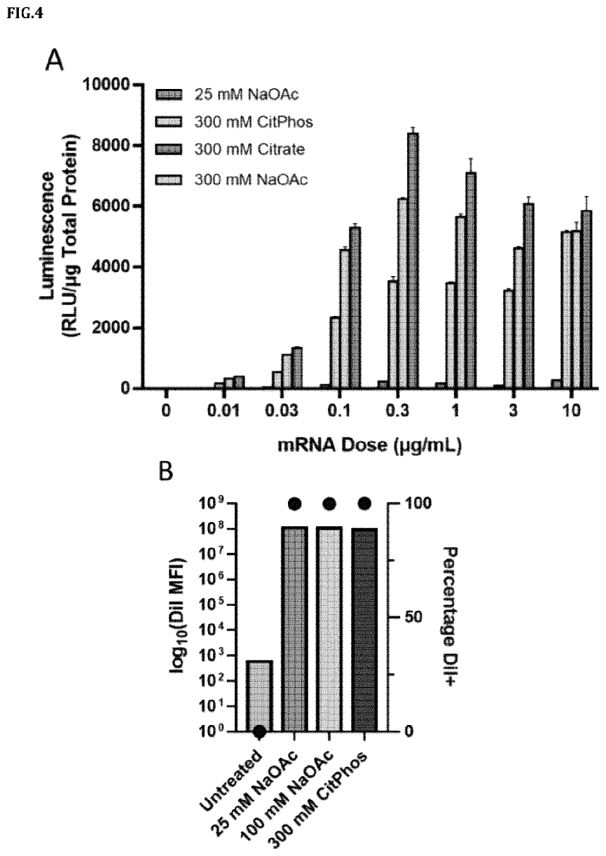

The average particle size for LNP were determined for the following compositions: KC2/DSPC/cholesterol/PEG-DMG formulated in citrate A) 25 mM NaOAc, B) 300 mM NaOAc, C) 300 mM Na-citrate Phosphate, and D) 300 mM Na-citrate. FIGS. 3A-D show that the particle sizes of LNPs prepared with 300 mM buffers were larger than those prepared with 25 mM NaOAc (FIGS. 3A-D).

The number of mRNA copies associated with blebs per LNP based on cross sectional area of LNP formulated in various buffers was next calculated.

It can be assumed that the smallest bleb structures contain only one copy of mRNA. Inspection of the cryo-TEM micrographs of FIGS. 1C and 1D obtained for the LNP mRNA prepared in the presence of 300 mM Cit-Phos or Na-citrate indicates a minimum cross-sectional area associated with the blebs of 230 nm². This value is consistent with the theoretical cross-sectional area of 200 nm² for a 2 kb mRNA in condensed, spherical form, assuming an mRNA diameter of 2 nm and a length per nucleotide of 0.33 nm¹⁹. Assuming that essentially all of the encapsulated mRNA exists in bleb structures for LNP mRNA systems prepared in the presence of 300 mM citrate (FIG. 1D) and that the area per monomer is 208 nm² it may be calculated that the average number of copies of mRNA encapsulated per LNP is 6.3 (FIG. 3E and Table 2 below). This may be compared to the theoretical value of an average of 5.5 copies of luc mRNA per LNP for the 60% of LNP that do contain mRNA. This theoretical estimate assumes an encapsulation efficiency of 80%, an N/P value of 6, a lipid density of 0.9 g/ml and an LNP diameter of 60 nm.

TABLE 2
Number of blebs observed, and mRNA copies associated with blebs per LNP calculated
based on cross sectional area of LNP formulated in various buffers

| | Buffer | | | |
|---|---|---|---|---|
| Parameters | 25 mM NaOAc | 300 mM NaOAc | 300 mM CitPhos | 300 mM Na-citrate |
| Number of Blebs | 3 | 4 | 125 | 144 |
| Minimum mRNA/LNP | 1 | 3.069 | 1.145 | 1.118 |
| Maximum mRNA/LNP | 4.987 | 14.84 | 24.67 | 48.72 |
| Mean ± SEM | 2.873 ± 1.157 | 7.904 ± 2.479 | 6.713 ± 0.3533 | 6.319 + 0.4605 |

### Example 4: The In Vitro and In Vivo Transfection Potencies of LNP mRNA Systems are Dramatically Improved by Formulating in 300 mM Na-Citrate Buffer

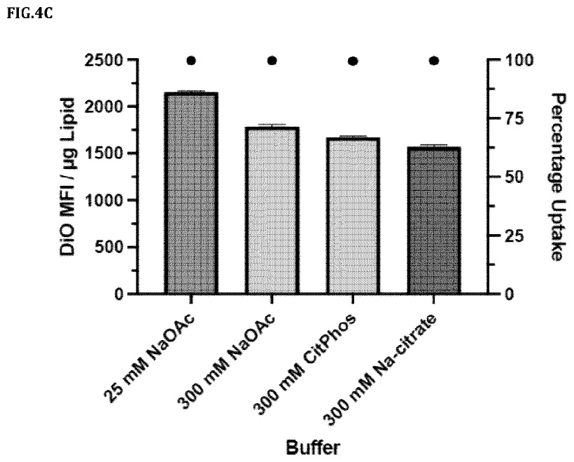

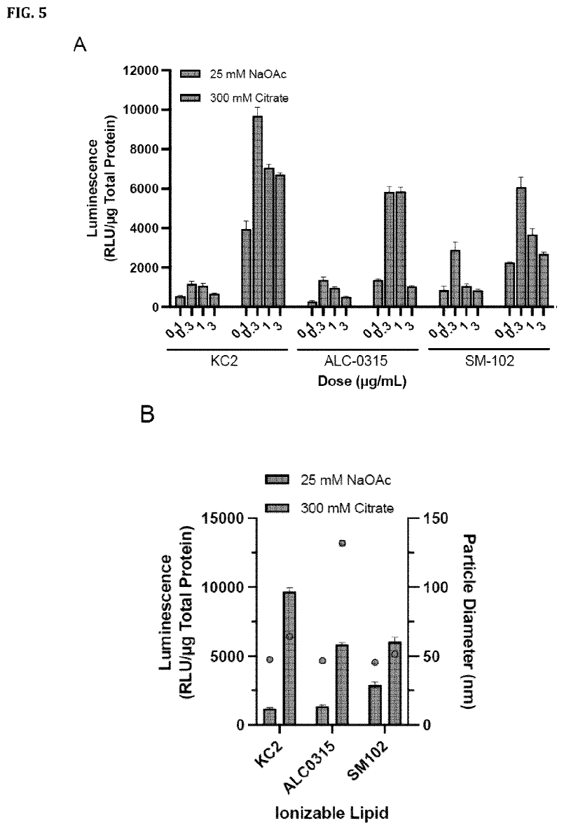

As noted in Example 1, LNPs with the sodium citrate buffer at 300 mM exhibited the highest transfection in HuH7 cells as measured by luminescence at 0.01-0.3 μg/mL (FIG. 1B). Higher doses were also examined (ranging from 1-10 μg/mL mRNA) and 300 mM citrate LNPs were again found to exhibit the highest transfection in HuH7 cells (FIG. 4A).

The improved transfection efficacy was not correlated to the cellular uptake, as no significant DiI uptake difference were observed between the lower and the higher ionic strength buffers (FIGS. 4B and 4C).

The next set of in vitro studies examined the relative improvement of transfection potencies induced by formulating in 300 mM Na-citrate for LNP luc mRNA systems containing different ionizable lipids. LNP luc mRNA formulations containing KC2, ALC-0315 and SM-102 were prepared using 300 mM Na-citrate as the pH 4 buffer and then incubated with HuH7 cells at varying concentrations. As shown in FIG. 5A, the most potent transfection system was the LNP luc mRNA system containing KC2 in which the 0.3 μg mRNA/mL concentration exhibited a 10-fold improvement in luminescence over that achieved by formulating with a 25 mM NaOAc buffer. It may be noted that the KC2 LNP mRNA system is nearly 2-fold more potent than LNP systems containing the advanced lipids ALC-0315 and SM-102 (FIG. 5B).

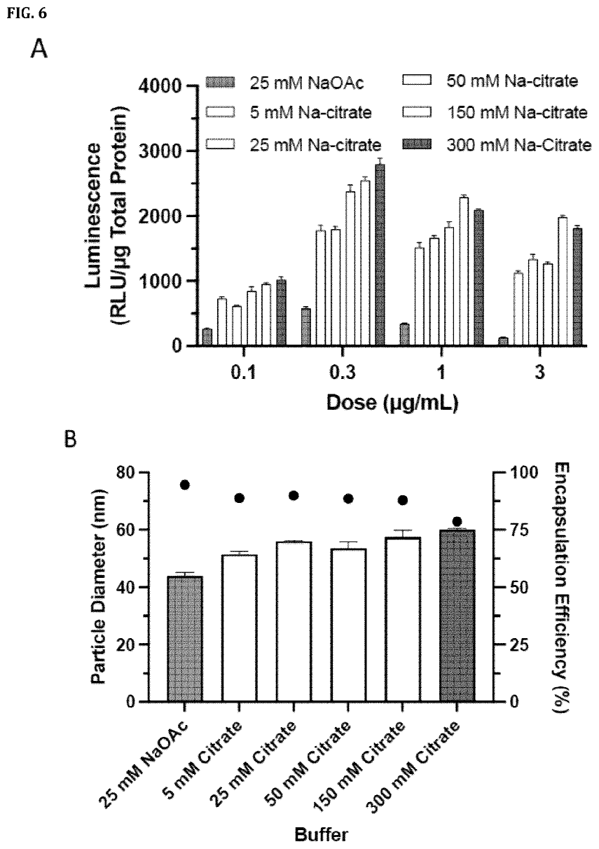

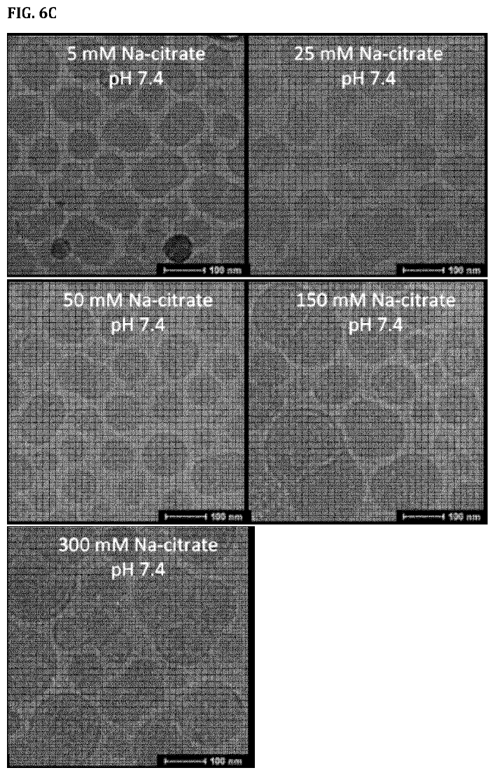

The properties of KC2 LNP mRNA particles produced by titration of the concentration of the pH 4 citrate buffer revealed that citrate buffers were highly effective for inducing bleb structure and enhancing transfection potency. Formulation with Na-citrate pH 4 buffers with concentrations as low as 5 mM led to significant induction of bleb structure and enhancement in transfection potency (see FIG. 6). However, the maximum in vitro transfection potency was still observed for LNP mRNA formulated using the 300 mM pH 4 citrate buffer (FIG. 6A). A strong correlation between induction of bleb structure and transfection potency is observed as the pH 4 citrate concentration is increased.

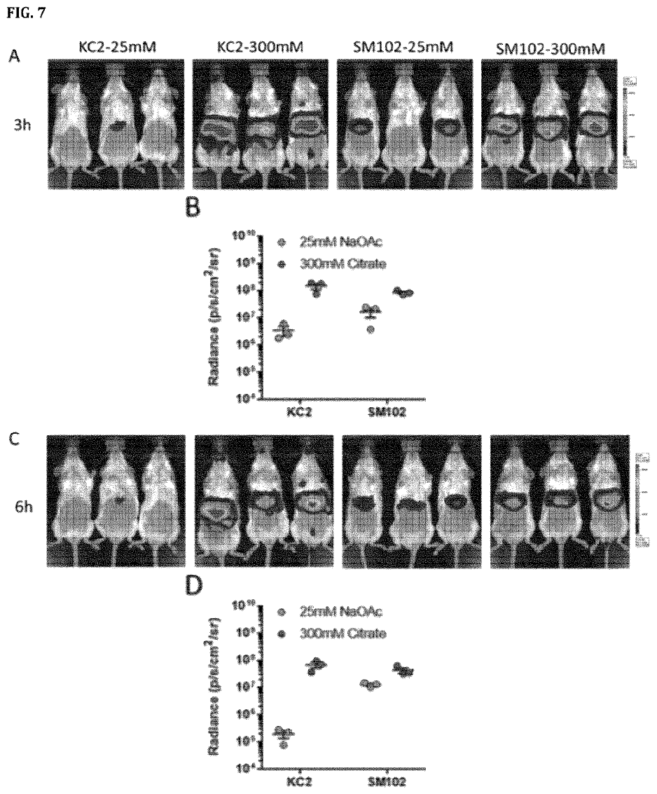

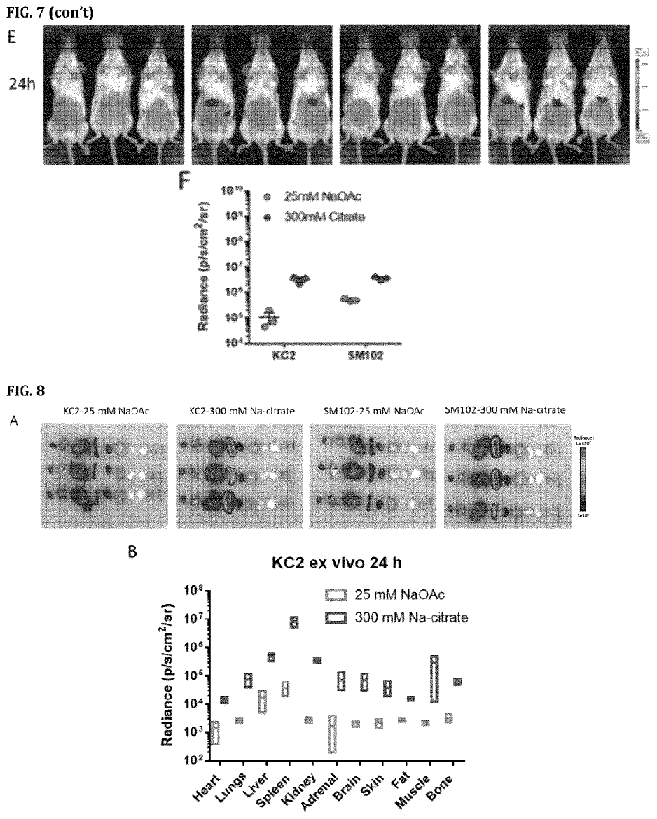

The inventors next determined whether the greatly improved in vitro transfection properties for LNP prepared using 300 mM pH 4 Na-citrate buffer concentrations also applied in vivo. LNP luc mRNA systems containing KC2 and SM-102 were formulated in either the 25 mM NaOAc pH 4 buffer or the 300 mM Na-citrate pH 4 buffer and were injected i.v into CD-1 mice at a dose level of 0.5 mg mRNA/kg and imaged at 3, 6 and 24 h. Bioluminescence was assayed at 3, 6 and 24 hours post-injection. As shown in FIG. 7 the LNP luc mRNA systems formulated using the 300 mM Na-citrate pH 4 buffer exhibited markedly improved gene expression (luciferase luminescence) in the liver and spleen.

The improvements in gene expression were particularly impressive for the KC2 LNP luc mRNA system. At 3 hr post injection, the abdominal bioluminescence observed following administration of the KC2 LNP luc mRNA system prepared using the 300 mM Na-citrate buffer was enhanced more than 40-fold compared to the signal achieved for the same system prepared using 25 mM NaOAc (FIGS. 7A-B, top). The SM-102 LNP luc mRNA systems prepared using the 300 mM Na-citrate buffer also gave rise to significantly higher luminescence than the same system prepared using 25 mM NaOAc, although the increase was less (5-fold).

The results for the KC2 LNP luc mRNA system prepared using the 300 mM pH 4 buffer were also notable at 6 hr post injection (FIGS. 7C-D, middle). The abdominal bioluminescence for mice receiving KC2 LNP luc mRNA prepared using the 300 mM pH 4 Na-citrate was more than 300-fold higher than in mice receiving KC2 LNP luc mRNA prepared using the 25 mM NaOAc buffer. Again, the KC2 LNP luc mRNA 300 mM Na-citrate system stimulated slightly higher luminescence than the corresponding SM-102 LNP mRNA system. Finally, significant bioluminescence remained at 24 hr for both the KC2 LNP luc mRNA and the SM-102 LNP luc mRNA systems prepared in 300 mM Na-citrate (FIGS. 7E-F, bottom).

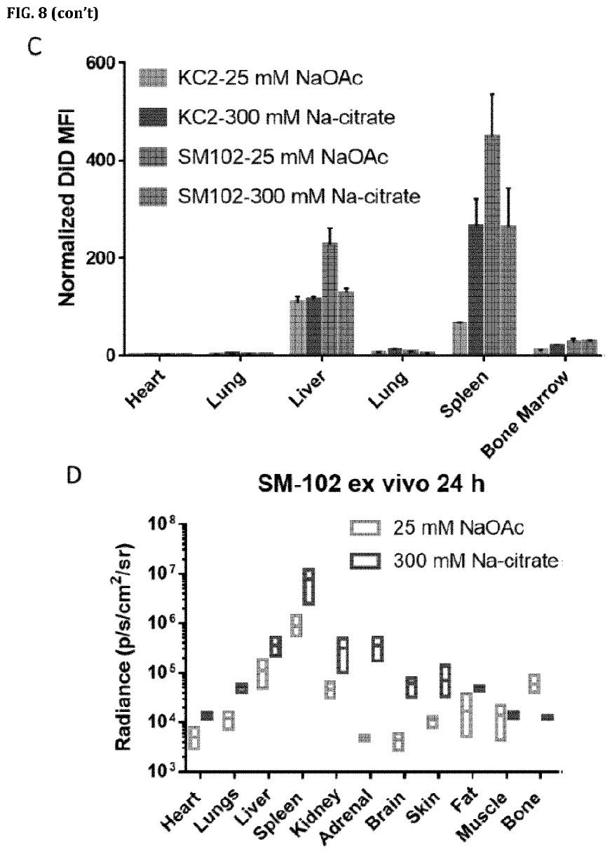

At 24 hr post-injection the mice were sacrificed, dissected and transfection assayed using ex-vivo IVIS imaging for heart, lungs, liver, kidney, adrenal, brain, skin, fat, muscle, and bone tissue. As shown in FIG. 8A, significant luminescence was detected in each organ/tissue. The signal from the spleen is particularly notable for the LNP mRNA systems prepared using the 300 mM (pH 4) sodium citrate buffer. The spleen luminescence observed following administration of KC2 LNP luc mRNA is more than 200-fold greater than for the same system prepared using the 25 mM NaOAc buffer at 24 hours (FIG. 8B). The fluorescence from the DiD labelled LNPs also revealed higher spleen signal of the 300 mM Na-citrate LNP over the 25 mM NaOAc LNP (FIG. 8C). The SM-102 LNP luc mRNA system prepared in the 300 mM Na-citrate buffer also exhibited a strong spleen signal but only a 9-fold increase compared to the same system prepared using the 25 mM NaOAc buffer (FIG. 8D).

### Example 5: LNP siRNA Systems Prepared Using High Buffer Concentrations Exhibit Improved Gene Silencing Properties In Vitro

The inventors next determined whether the substantial benefits of formulating LNP mRNA systems using high concentrations of buffers (pH 4) also extends to improving the gene silencing properties of LNP siRNA systems. KC2 LNPs were formulated with siRNA against luciferase (siLuc) using 25 mM NaOAc, 300 mM NaOAc, 300 mM Na-citrate phosphate and 300 mM Na-citrate pH 4 buffers before being dialyzed into PBS. As shown in Table 1 (above in Example 2) excellent encapsulation efficiencies were achieved for all formulations. As opposed to LNP containing mRNA, the size of formulations prepared at high pH buffer concentrations tended to decrease for the LNP containing siRNA (Table 1).

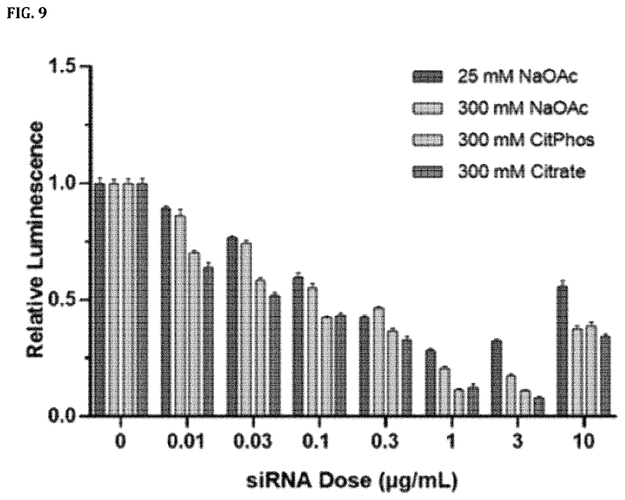

The in vitro gene silencing properties of the LNP siLuc formulations were assayed by incubating various concentrations of LNP with 22Rv1 cells expressing luciferase. As shown in FIG. 9, LNPs formulated with high concentrations of buffer improved gene silencing properties by ˜20% across all siRNA doses tested.

### Example 6: LNP-mRNA Stability, mRNA Integrity and Wheat Germ Extract Assay

Without being bound by theory, the reason why the LNP mRNA systems exhibiting bleb structures also exhibit enhanced transfection properties both in vitro and in vivo could arise from a number of sources. One possibility concerns the integrity of the mRNA. It is believed that mRNA surrounded by a lipid bilayer is significantly more stable in the presence of serum than mRNA in the typical solid core LNP mRNA system. As noted, the mRNA in LNP mRNA systems exhibiting blebs is protected from the outer medium by a lipid bilayer, potentially conferring improved stability.

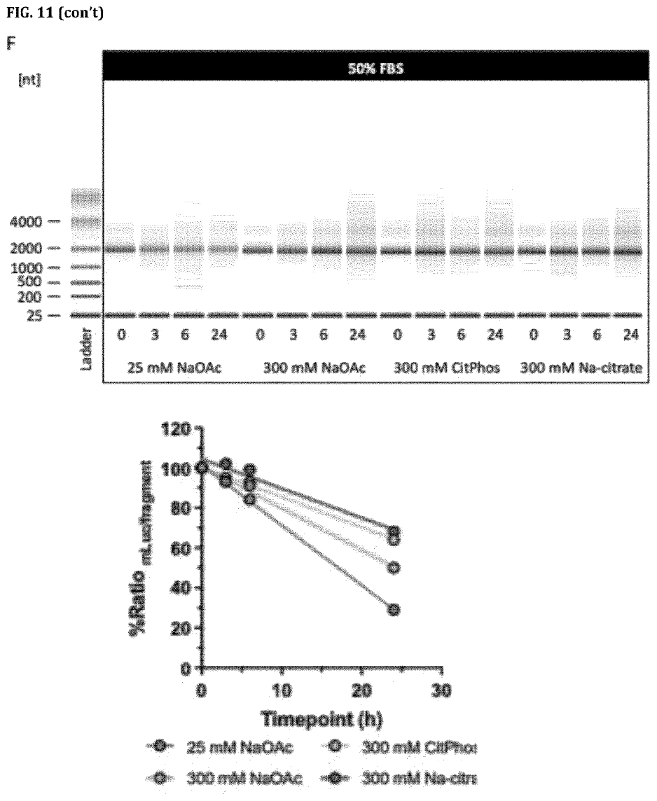

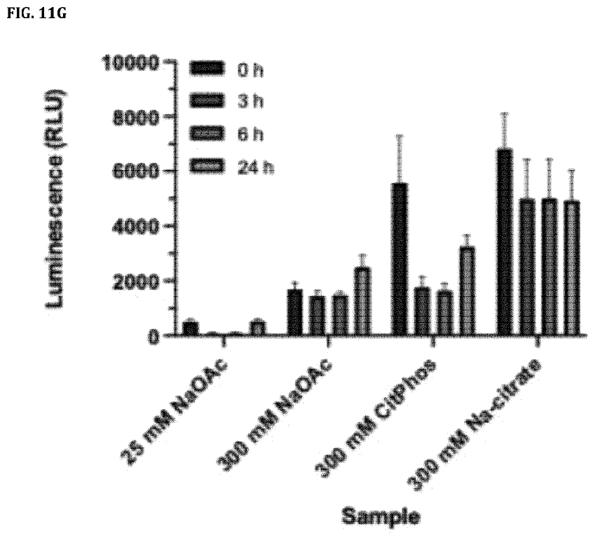

In order to determine whether this is the case, the stability of mRNA prepared in LNP formulated in the 25 mM NaOAc pH 4 buffer in the presence of serum was compared to the stability of mRNA in LNP mRNA prepared using the 300 mM Na-citrate buffer. As shown in FIG. 11, a BioAnalyzer™ analysis of the mRNA integrity suggested that the mRNA formulated in LNP using the 300 mM citrate pH 4 buffer is significantly more stable than mRNA formulated in the 25 mM NaOAc buffer. In order to determine how this may affect the translation capabilities of the mRNA, a cell free wheat germ assay on mRNA extracted from LNP formulated in 25 mM NaOAc and high concentrations of NaOAc, CitPhos and Na-citrate was performed. Remarkably, the mRNA extracted from LNP mRNA systems formulated in the 300 mM Na-citrate pH 4 buffer was 10-fold or more translation competent than mRNA extracted from LNP formulated in the 25 mM NaOAc buffer.

### Example 8: No Significant Toxicity was Observed Between Bleb or Non-Bleb LNP Luc mRNA Systems

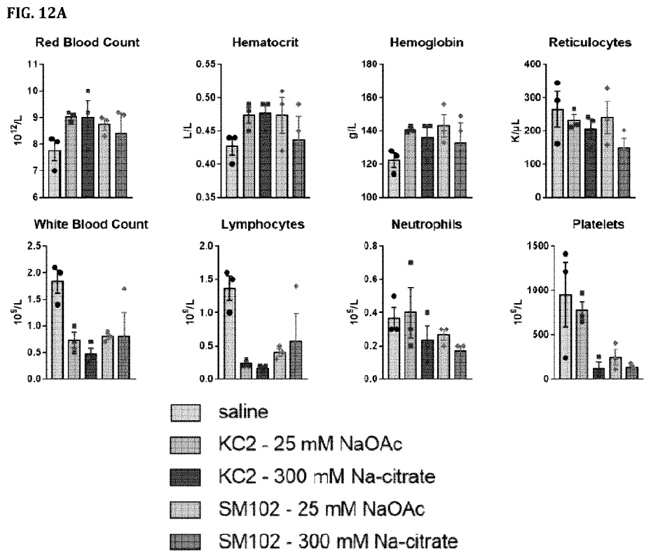

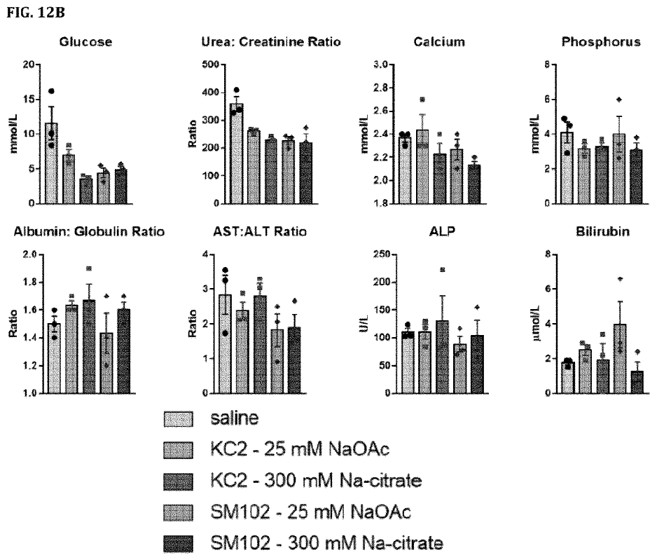

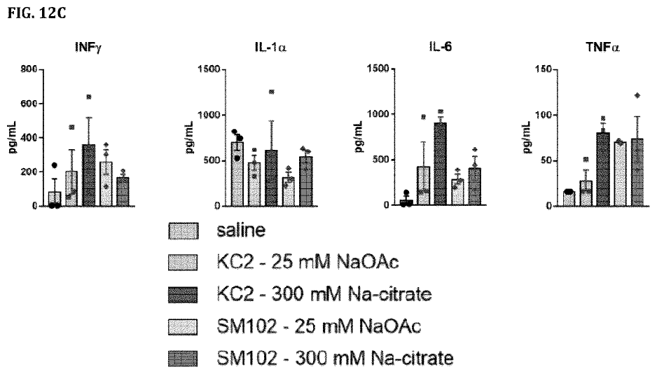

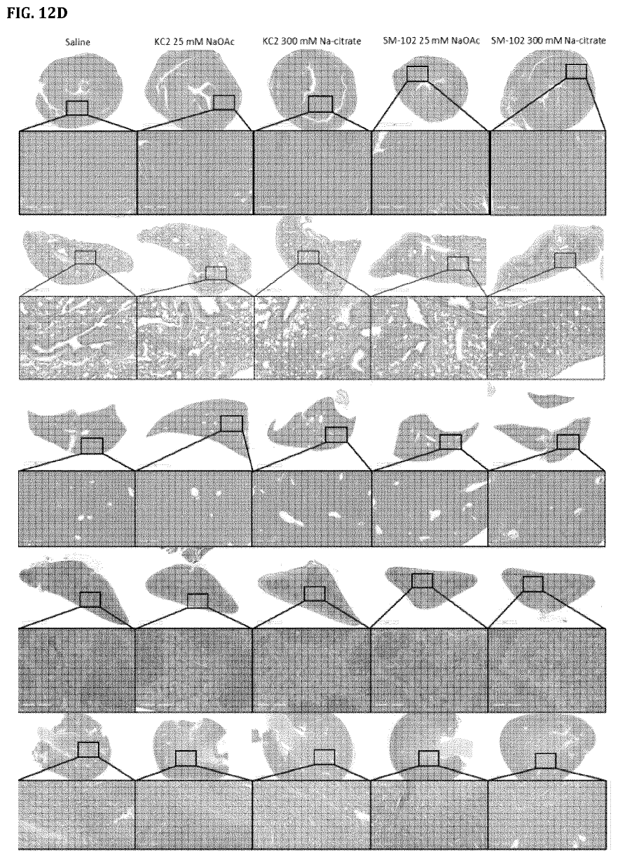

Experiments were conducted to establish that the enhanced potency of the LNP mRNA systems prepared in 300 mM citrate pH 4 buffers did not cause toxicity as compared to systems prepared using the 25 mM NaOAc buffer. CD-1 mice were injected i.v. with LNP luc mRNA at an mRNA dose of 1.5 mg/kg, three times higher than used for the gene expression studies of FIG. 7. No significant difference in hematology, biochemistry or cytokine profile was observed 24 hr post-injection between the two LNP formulations. Organs such as heart, lung, liver, spleen and kidney were excised for hematoxylin and eosin (H&E) staining. No differences were observed between the control and treatment groups (FIG. 12A-D).

The detailed description and examples are intended to illustrate specific embodiments of the invention but are in no way intended to limit its scope thereof.

### REFERENCES

1 Cheng, M. H. Y., Brimacombe, C. A., Verbeke, R. & Cullis, P. R. Exciting Times for Lipid Nanoparticles: How Canadian Discoveries Are Enabling Gene Therapies. Mol Pharm 19, 1663-1668 (2022). https://doi.org: 10.1021/acs.molpharmaceut.2c00365
2 Akinc, A. et al. The Onpattro story and the clinical translation of nanomedicines containing nucleic acid-based drugs. Nat Nanotechnol 14, 1084-1087 (2019). https://doi.org: 10.1038/s41565-019-0591-y
3 Polack, F. P. et al. Safety and Efficacy of the BNT162b2 mRNA Covid-19 Vaccine. N Engl J Med 383, 2603-2615 (2020). https://doi.org: 10.1056/NEJMoa2034577
4 Musunuru, K. et al. In vivo CRISPR base editing of PCSK9 durably lowers cholesterol in primates. Nature 593, 429-434 (2021). https://doi.org: 10.1038/s41586-021-03534-y
5 Rurik, J. G. et al. CAR T cells produced in vivo to treat cardiac injury. Science 375, 91-96 (2022). https://doi.org: 10.1126/science.abm0594
6 Semple, S. C. et al. Rational design of cationic lipids for siRNA delivery. Nat Biotechnol 28, 172-176 (2010). https://doi.org: 10.1038/nbt.1602
7 Semple, S. C. et al. Efficient encapsulation of antisense oligonucleotides in lipid vesicles using ionizable aminolipids: formation of novel small multilamellar vesicle structures. Biochim Biophys Acta 1510, 152-166 (2001). https://doi.org: 10.1016/s0005-2736 (00) 00343-6
8 Zimmermann, T. S. et al. RNAi-mediated gene silencing in non-human primates. Nature 441, 111-114 (2006). https://doi.org: 10.1038/nature04688
9 Jayaraman, M. et al. Maximizing the potency of siRNA lipid nanoparticles for hepatic gene silencing in vivo. Angew Chem Int Ed Engl 51, 8529-8533 (2012). https://doi.org: 10.1002/anie.201203263
10 Brader, M. L. et al. Encapsulation state of messenger RNA inside lipid nanoparticles. Biophys J 120, 2766-2770 (2021). https://doi.org: 10.1016/j.bpj.2021.03.012

1. A method for preparing a nucleic-acid lipid nanoparticle, the method comprising:
(i) combining an aqueous phase comprising the nucleic acid with an organic solvent-lipid mixture comprising lipids, wherein the lipids comprise an ionizable lipid and at least one helper lipid and optionally a hydrophilic-polymer lipid conjugate;
wherein the aqueous phase comprises a carboxylate-containing buffer having a concentration of at least 100 mM and has an aqueous phase pH that is lower than a pKa of the ionizable lipid such that the ionizable lipid is substantially charged;
wherein the lipid nanoparticle is formed during or subsequent to the combining; and
(ii) exchanging a solution external to the lipid nanoparticle with a higher pH solution, thereby producing the nucleic-acid lipid nanoparticle that comprises one or more bleb compartments in which the nucleic acid is dissociated from the ionizable lipid.

2. The method of claim 1, wherein the higher pH solution has a pH of at least 6.0.

3. The method of claim 2, wherein the higher pH solution has a pH of at least 6.25.

4. The method of claim 3, wherein the higher pH solution has a pH of at least 6.50.

5. The method of claim 4, wherein the higher pH solution has a pH of at least 6.60.

6. The method any one of claims 1 to 5, wherein the pH of the aqueous phase in step (i) is between 3.0 and 8.0.

7. The method of claim 6, wherein the pH of the aqueous phase in step (i) is between 3.25 and 6.5.

8. The method of claim 7, wherein the pH of the aqueous phase in step (i) is between 3.50 and 6.0.

9. The method of any one of claims 1 to 8, wherein the aqueous phase in step (i) comprises a buffer at a concentration of between 100 and 600 mM.

10. The method of claim 9, wherein the aqueous phase comprises the buffer at a concentration of between 125 and 575 mM.

11. The method of claim 10, wherein the aqueous phase comprises the buffer at a concentration of between 150 and 550 mM.

12. The method of claim 11, wherein the aqueous phase comprises the buffer at a concentration of between 175 and 525 mM.

13. The method of claim 12, wherein the aqueous phase comprises the buffer at a concentration of between 200 and 500 mM.

14. The method of claim 13, wherein the aqueous phase comprises the buffer at a concentration of between 250 and 475 mM.

15. The method of any one of claims 1 to 14, wherein the buffer is biocompatible.

16. The method of any one of claims 1 to 15, wherein the buffer is a salt or acid of acetate, citrate, phosphate or sulfonate.

17. The method of claim 16, wherein the buffer is a salt of citrate.

18. The method of claim 17, wherein the buffer is a salt of citrate.

19. The method of any one of claims 1 to 18, wherein the ionizable lipid comprises an ionizable nitrogen moiety.

20. The method of any one of claims 1 to 19, wherein the nucleic acid-lipid nanoparticle produced in step (ii) comprises a hydrophobic compartment is disposed between two leaflets of a bilayer that forms a lipid layer of the nanoparticle.

21. The method of claim 20, wherein the one or more bleb compartments are surrounded on one side by an inner leaflet of the lipid layer enclosing the hydrophobic compartment and on another side by a portion of the lipid layer that forms an external bilayer.

22. The method of any one of claims 1 to 21, wherein the one or more bleb compartments are convex-shaped bilayer protrusion as visualized by cryo-TEM.

23. The method of any one of claims 1 to 22, wherein the one or more bleb compartments are hemispherical.

24. The method of any one of claims 1 to 23, wherein the lipid nanoparticle produced in step (ii) has a transfection efficiency that is at least 2-fold greater than a transfection efficiency of a baseline solid core lipid nanoparticle having the same lipids and nucleic acid and produced by an identical method apart from buffer in step (i) being 25 mM sodium acetate at pH 4.0.

25. The method of any one of claims 1 to 24, wherein the nucleic acid is mRNA, DNA vector, siRNA or anti-sense oligonucleotide.

26. The method of any one of claims 1 to 24, wherein the nucleic acid is part of a gene editing system.

27. The method of any one of claims 1 to 26, wherein the combining is carried out in a mixing device.

28. The method of claim 27, wherein the mixing device is a T-mixer, a herringbone micromixer, a toroidal mixer or a multi-inlet vortex mixer.

29. The method of claim 28, wherein the mixing device is a T-junction mixer comprising pumps to introduce respective streams of the organic solvent lipid phase and the aqueous phase comprising the nucleic acid at a mixing point.
